(19) United States
(12) Patent Application Publication
(10) Pub. No.: US 2024/0226024 A1
(43) Pub. Date: Jul. 11, 2024

(54) SYNERGISTIC EFFECTS ON WEIGHT LOSS, IMPROVED QUALITY OF LIFE AND REDUCED GASTRO-INTESTINAL SIDE EFFECTS WITH A COMPOSITION OF ORLISTAT AND ACARBOSE

(71) Applicant: Empros Pharma AB, Solna (SE)

(72) Inventors: Jan Stefan Persson GRUDÉN, Uppsala (SE); Anders FORSLUND, Uppsala (SE); Ulf HOLMBÄCK, Uppsala (SE); Jan Arvid SÖDERHALL, Spånga (SE); Göran ALDERBORN, Uppsala (SE)

(73) Assignee: Empros Pharma AB, Solna (SE)

(21) Appl. No.: 18/562,761

(22) PCT Filed: May 20, 2022
(86) PCT No.: PCT/EP2022/063746

§ 371 Date: Nov. 20, 2023

(30) Foreign Application Priority Data
May 21, 2021 (EP) 21175174.8
Jan. 31, 2022 (EP) 22154200.4

(51) Int. Cl. A61K 9/50; A61K 31/365; A61K 31/702; A61P 3/04

(52) U.S. Cl. CPC A61K 9/5084; A61K 9/5047; A61K 9/5078; A61K 31/365; A61K 31/702; A61P 3/04

(57) ABSTRACT

The invention relates to a composition of orlistat and acarbose for use in improving quality of life. The combination is administered orally to obtain a synergistic effect. The synergistic effect is obtained after 13 weeks or more such a after 14 weeks or more. after 15 weeks or more. after 16 weeks or more. after 17 weeks or more. after 18 weeks or more. after 19 weeks or more. after 20 weeks or more. after 21 weeks or more. after 22 weeks or more. after 23 weeks or more. after 24 weeks or more. after 25 weeks or more of after 26 weeks or more of treatment. The synergistic effect gives improved weight loss.

### FIELD OF THE INVENTION

The present invention relates to the use of a composition of orlistat and acarbose in medicine to obtain a synergistic effect with respect to weight loss and reduced gastrointestinal side effects. The invention also relates to the use of such a composition to obtain an improvement in quality of life compared with baseline or placebo. The use in medicine is to reduce body weight, i.e., for use in situations where a reduction in body weight is desired, but as seen from the examples herein a marked improvement in quality of life is obtained. A composition of the invention may be used in the treatment of overweight and obesity; type 2 diabetes; elevated blood glucose level (such as impaired glucose tolerance), polycystic ovarian syndrome; disorders of lipoprotein metabolism and other lipidemia (such as hyperglyceridemia); nonalcoholic fatty liver disease (NAFLD); nonalcoholic steatohepatitis; metabolic syndrome; or pre-operative weight loss. The composition may also be used for cosmetic purposes.

### BACKGROUND

The worldwide prevalence is estimated to be 1.5 billion overweight and 500 million obese individuals. Overall, more than one out of ten of the world's adult population is obese. In 2010, more than 40 million children under five were overweight. Once considered a high-income country problem, overweight and obesity are now on the rise in low- and middle-income countries, particularly in urban settings. Overweight and obesity are the fifth leading risks for global deaths. At least 2.8 million adults die each year globally as a result of being overweight or obese. In addition, 44% of the diabetes burden, 23% of the ischemic heart disease burden and between 7% and 41% of certain cancer burdens are attributable to overweight and obesity. In June 2013, the American Medical Association officially recognized obesity as a disease.

There is a great concern globally of this serious health issue, but different strategies have not been successful to reverse the obesity trends among the global population. Neither has the awareness for healthier diet and increased physical activity proved particularly effective. There exist several potential explanations such as: the absence of access to healthy, affordable foods or safe places for physical activity, particularly in lower-income neighbourhoods and communities; the inferiority of freshly prepared foods vs. fast foods or pre-packaged foods in terms of preservation, portability, and palatability; the marketing of mostly unhealthy products by the food and beverage industry; and modern cultural habits that increase sedentary behaviours, degrade eating cadences and locations, and incur excess stress levels and sleep debt. Life-style intervention affecting dietary intake and energy expenditure are important, however, often not enough. It is obvious that obesity should be considered as a chronic, incurable disease, which needs better drug products for a successful treatment. Therefore, there is a need for a novel safe and efficient medical treatment.

Type 2-diabetes is growing epidemically, and this rise is closely associated with obesity. Type 2-diabetes has multiple manifestations and sub-optimal treatment is associated with progressive beta-cell failure. Although lifestyle measures, including eating habits and physical activity, should be first-line treatment, success is difficult to achieve, and pharmaceutical intervention is almost always required. Before manifest type 2-diabetes is diagnosed, the patients usually have a period of impaired glucose tolerance. If this impaired glucose tolerance, which may precede or follow weight gain, is correctly treated, the progression towards diabetes might be halted or averted. Current treatment options are limited to lifestyle changes, or secondly metformin. Hence, there is a need for a novel safe and efficient medical treatment.

Yet another indication for this invention would be treatment of overweight/obesity in association with Polycystic Ovary Syndrome (PCOS). Polycystic ovary syndrome (PCOS) is one of the most common endocrine disorders among females and produces symptoms in 5% women of reproductive age (conservative figure). One of the most common immediate symptoms is insulin resistance. This insulin resistance is often associated with obesity, type 2 diabetes, and high cholesterol levels. Current recommended pharmacological treatment (in addition to contraceptives) of the obese and/or glucose impaired PCOS patients is limited to metformin, although current guidelines state that the evidence base is not strong. Other insulin sensitizers, for example thiazolidinediones, have unwanted risk/benefit ratio and are not recommended. For the PCOS patients, there is a clinical need for a drug that safely both decreases weight and improves glucose tolerance.

Nonalcoholic steatohepatitis (NASH) is liver inflammation and damage caused by a buildup of fat in the liver. NASH affects 2 to 5 percent of Americans. An additional 10 to 20 percent of Americans have fat in their liver, but no inflammation or liver damage, a condition called “fatty liver.” or NAFLD. Both NASH and NAFLD are becoming more common, possibly because of the greater number of Americans with obesity. Currently, no specific therapies for NASH exist, except for lifestyle interventions, so there exists an unmet clinical need.

According to the new International Diabetes Federation (IDF) definition, for a person to be defined as having the metabolic syndrome the person must have:

Central obesity plus any two of the following four factors:
raised triglyceride (TG) level or specific treatment for this lipid abnormality
reduced high-density lipoprotein (HDL) cholesterol or specific treatment for this lipid abnormality
raised blood pressure or treatment of previously diagnosed hypertension
raised fasting plasma glucose or previously diagnosed type 2-diabetes

The present inventors postulate that the proposed product will directly or indirectly affect most of the components of the metabolic syndrome, mainly decreasing weight, improving glucose control, which in turn will lead to improved hepatic fat metabolism with decreased triglycerides concentration. The product is expected to also have direct effect on triglyceride concentration.

### Current Treatment Options for Obesity and Overweight

Several pharmacological principles have been considered for treatment of obesity or overweight including increasing energy expenditure (stimulants), suppressing caloric intake (anorectic agents), limit nutrient absorption and modulating insulin production and/or action. Four centrally-acting noradrenergic agents (phentermine, diethylpropion, phendimetrazine, benzphetamine) are FDA-approved for usually less than 12 weeks management of obesity. All were approved before the necessity of long-term treatment for obesity was established. In addition, none were required to meet the current efficacy benchmarks for weight loss relative to placebo (mean weight loss ≥5% more than that of the placebo group or proportion of drug-treated subjects who lose ≥5% of initial weight is ≥35% and approximately double the proportion who lose ≥5% in the placebo group). Drugs for weight management that are approved for long-term usually result in, on average, an additional weight loss relative to placebo ranging from ˜3% for orlistat and lorcaserin to 9% for phentermine/topiramate-ER at one year. Already in 2005, the stimulants, including dinitrophenol, amphetamine and ephedra, were abandoned. Among anorectic agents sibutramine was on the market for a few years before adverse effects led to its removal, together with the short-lived appetite suppressor Rimonabant. Lorcaserin is a selective serotonin 2C (5HT2c) receptor agonist that was anticipated to recapitulate the weight loss effects of fenfluramine without its adverse cardiac effects. Lorcaserin decreased body weight modestly, by about 3.2 kg (˜3.2% of initial body weight) more than placebo Among patients with diabetes, lorcaserin treatment led to lower body weight and improved glycated hemoglobin concentrations. Liraglutide (Saxenda®; liraglutide injection) was approved (both by EMA and FDA) as a treatment option for chronic weight management in addition to a reduced-calorie diet and physical activity. The drug is approved for use in adults with a body mass index (BMI) of 30 or greater (obesity) or adults with a BMI of 27 or greater (overweight) who have at least one weight-related condition such as hypertension, type 2 diabetes, or high cholesterol (dyslipidemia). GLP-1 analogues (such as liraglutide and exenatide) have initially been used as diabetes type-2 medication, but successful weight loss trials have been performed where patients lost 8 kg more after one year on the highest dose of liraglutide; compared to the placebo group which lost 2 kg. However, safety concerns exist regarding these drugs, chiefly regarding suggested increased risk of developing pancreatic cancer. The FDA approves the use of liraglutide but encourages both prescribers and patients to report possible side effects.

During the last 20 years, about 10 different drugs have been put out on the market, only to be withdrawn within a few years. The current alternatives include attempts to limit nutrient (lipids) absorption (orlistat), and perhaps to use compounds affecting insulin (see below). In conclusion: the available pharmaceutical products based on a single unit that possesses a positive benefit-risk ratio for this patient group are very limited.

Currently, orlistat (Xenical®) and liraglutide (Saxenda®) are the only available antiobesity drugs worldwide. Orlistat (Xenical®) is available both in prescription (120 mg) and over-the-counter (60 mg) strength and is given by the oral route. Orlistat is a semi-reversible and local inhibitor of gastric and pancreatic lipases in the GI tract and acts as an antiobesity drug by preventing intestinal absorption of dietary fats (i.e., reducing energy intake). The fraction of the dose absorbed of the highly lipophilic orlistat (log P 8.5) is low (<3%) and accordingly the plasma exposure is low (<5 ng/ml). Today, orlistat is available in a conventional relative rapid release oral dosage form. However, orlistat, although safe, is associated with some side-effects that severely hamper compliance. In clinical trials, about 25% or more of the patients complain about GI side-effects including diarrhea, oily spotting and fecal urgency. This, in conjunction with the rather modest effect on weight (Best case scenario: 10% relative weight loss versus placebo 6% relative weight loss, makes orlistat in this conventional and relative rapid release dosage form unattractive for the vast majority of obese patients. However, in a recent report FDA clearly stated that orlistat is safe and has clinical benefit. Acarbose (Glucobay®) is a competitive α-glucosidase and pancreatic α-amylase inhibitor, which inhibits the hydrolysis of oligosaccharides during GI luminal digestion of a meal. Acarbose has hydrophilic properties (log P −8.1) and consequently low intestinal permeability, low fraction dose absorbed (<5%), low bioavailability and systemic exposure of acarbose. Acarbose, available in conventional immediate release dosage form, is currently used as a diabetic drug, mainly in Asia, but only scarcely in Western countries. It has not been approved for treatment of obesity.

As with orlistat, a large part of the patients using acarbose reports GI tolerability problems (mainly flatulence, diarrhea as well as GI and abdominal pains), which limits its current clinical use in western countries.

There are currently two other α-glucosidase inhibitors on the market, miglitol and voglibose. Miglitol is FDA approved and available in several countries, whereas voglibose is approved only in Japan. Acarbose, miglitol and voglibose lowers HbA1c to more or less the same extent, with slightly different side effect. Miglitol is absorbed to 100% and is excreted though the kidneys; whereas voglibose is, in similarity to acarbose, only negligibly absorbed. Voglibose, most probably due to its low dose (0.2 mg voglibose/meal is a common dose) shows lower frequency of GI side effects compared to acarbose; but does not decrease rate of gastric emptying. So far available studies indicate that all three α-glucosidase inhibitors are safe with no systemic effects. There is also a plethora (>1200 compounds) of identified plant compounds that show varying α-glucosidase inhibitory effects. Acarbose stands out as it is by far the most clinically used and investigated compound, is approved worldwide and its patent has expired.

There is currently no other lipase inhibitor approved for treatment of obesity, with the possible exception of cetilistat. Cetilistat has been shown to have led to similar weight reduction as orlistat, but with much lower frequency of side effects. Cetilistat is currently only approved in Japan. There are also some lipase inhibitors from plants, where a few can be bought as OTC-drugs. Thus, the list of potential lipase inhibitors is very short.

As it appears from the discussion above, there is a need for developing compositions and administration regimes for treatment of obesity or overweight that is more effective, but also has reduced side effects and improved tolerability compared with the products on the market today, especially compared with Xenical® tablets. The present invention is a development of the invention described in Applicant's patent application published as WO 2016/097170. It relates to a modified release composition of acarbose and orlistat present in the composition in three different parts with different release pattern. Surprisingly, treatment results over 6 months of the clinical study with this new composition have shown that a synergistic effect of orlistat and acarbose is obtained which means that reduction of body weight obtained by treatment with this new composition of orlistat and acarbose is increased compared with what would have been expected based on an effect obtained by adding the effects from orlistat and acarbose.

### SUMMARY OF THE INVENTION

The present invention relates to a composition comprising orlistat and acarbose for use in improving quality of life in obese or overweight subjects. The subjects are humans. Typically, such a composition is used in the treatment of obese or overweight subjects, and—apart from a reduction in body weight—the treatment leads to an improvement in quality of life.

Typically, the obese or overweight subjects have a BMI of 25 kg/m² or more such as 27 kg/m², 29 kg/m² or more or 30 kg/m² or more. Overweight subjects have a BMI of 25 kg/m² or more such as from 25 kg/m² to less than 30 kg/m², and obese subjects have a BMI of 30 kg/m² or more.

A composition for use according to the invention normally comprises orlistat and acarbose in a weight ratio of from 2:1 to 4:1 such as 180/60, 150/50 or 120/40 orlistat/acarbose

The improvement in quality of life is assessed by a questionnaire including questions to following domains: physical functioning, bodily pain, role limitations due to physical health problems, role limitations due to personal or emotional problems, emotional well-being, social functioning, energy/fatigue, and general health perception. A suitable questionnaire and generally used in clinical trial is RAND-36 health survey. Other suitable questionnaires are SF-36, Impact of Weight on Quality of Life (IWQOL), Obesity and Weight Loss Quality of Life (OWLQOL) or similar health surveys.

By the questionnaire, a mean overall health transition score can be measured. In general, the improvement in quality of life is observed when the mean overall health transition score is 10 points or more such as 12 points or more, 14 points or more, 15 points or more, 16 points or more compared with baseline.

In general, the improvement is in at least 5 of the following domains: physical functioning, bodily pain, role limitations due to physical health problems, role limitations due to personal or emotional problems, emotional well-being, social functioning, energy/fatigue, and general health perception. In particular, an improvement is measured as increased physical functioning and/or general health. The improvement in mean score is increased by 10 or more compared to baseline.

During in the treatment period the composition comprising orlistat and acarbose is administered one, two or three times daily. In general, the treatment period is at least 2 weeks such as from about 2 weeks to about 1 year such as from about 2 weeks to about 9 months, from about 2 weeks to about 6 months, from about 2 weeks to about 5 months, from about 2 weeks to about 4 months, from about 2 weeks to about 3 months, from about 2 weeks to about 2 months. In particular, the treatment period is 26 weeks or more.

During the treatment period the subject is on a regular basis requested to answer the questionnaire. The time period between answers is given to the questionnaire depends on the treatment period, but typically it is at least 4 weeks such as at least 6 weeks, at least 8 weeks or at least 26 weeks.

The improvement of quality of life is achieved regardless of achieved weight loss of said subject during treatment. In particular, the improvement of quality of life may be achieved in combination with an achieved weight loss of said subject during treatment of at least 5%.

Typically, a daily dose of orlistat in the treatment period is from 30 mg to 540 mg or more, from 30 mg to 450 mg or more such as from 60 mg to about 450 mg or more, from 90 mg to about 450 mg or more, from about 120 mg to 450 mg or more, from about 150 mg to about 450 mg or more, from 180 mg to 450 mg or more such as from 180 mg to 450 mg, from 270 mg to 450 mg, from 360 mg to 450 mg for an adult is 270 mg or more 360 mg or more or 450 mg or more.

Typically, a daily dose of acarbose in the treatment period is from 10 mg to about 180 mg or more, from 10 mg to about 150 mg such as from 20 mg to about 150 mg, from 30 mg to about 150 mg, from 40 mg to about 150 mg, from 50 mg to about 150 mg, from 60 mg to about 150 mg or more such as from 90 mg to 150 mg, 90 mg or more, 120 mg or more or 150 mg or more.

In embodiments, said composition comprises 90 mg orlistat/30 mg acarbose, 120 mg orlistat/40 mg orlistat, 150 mg orlistat/50 mg acarbose or 180 mg orlistat/60 mg acarbose.

Suitable compositions for use according to the invention are designed for oral administration and are designed to release orlistat and acarbose at suitable locations in the gastrointestinal tract. To achieve this goal a composition suitable for may comprise granules, spheres or pellets.

As discussed herein, suitable compositions are those, wherein orlistat is in micronized form, i.e., with an average particle size below 50 microns such as below 20 microns such as below 10 microns. Moreover, the present inventors have found that orlistat and enteric polymers may react in an undesired manner and, accordingly, when an enteric polymer is present in the composition, orlistat should be protected from direct contact with the enteric polymer. This may be done by use of a protective polymer such as a polymer selected from cellulose, cellulose derivatives, and hydroxypropyl methylcellulose.

Specifically, a composition is an oral modified release composition comprises three or four different individual parts with different release pattern:
a) a first part, G1, comprising from about 45% w/w to about 65% w/w such as from about 50% w/w to about 65% w/w, from about 55% w/w to about 65% w/w or about 60% w/w of the total dose of acarbose,
b) a second part, G2A, comprising from about 35% w/w to about 55% w/w such as from about 35% w/w to about 50% w/w, from about 35% w/w to about 45% w/w or about 40% w/w of the total dose of acarbose,
c) a third part, G2B, comprising from about 50% w/w to about 85% w/w such as from about 55% w/w to about 80% w/w, from about 60% w/w to about 80% w/w, from about 65% w/w to about 75% w/w, from about 68% w/w to about 75% w/w, from about 72% w/w to about 73% w/w such as about 72.2% w/w of the total dose of orlistat, and
d) a fourth part, G3, comprising from about 15 to about 50% w/w such as from about 20% w/w to 40% w/w, from about 25% to about 35% w/w, from about 25% to about 32% w/w, from about 27% w/w to about 28% w/w or about 27.8% w/w of the total dose of orlistat,
and the total concentration of acarbose and orlistat, respectively, is 100% w/w,
wherein—when the composition contains three parts, the three parts are i) G1, ii) G2 wherein G2A and G2B are combined, and iii) G3.

Such a composition is designed in such a manner that
i) part G1 is designed to release a part of the total dose of acarbose in the stomach,
ii) part G2A is designed to release a part of the total dose of acarbose in duodenum and jejunum; the release should be relatively fast, as acarbose should be available to exert their effect in duodenum and jejunum,
iii) part G2B is designed to release a part of the total dose of orlistat in duodenum and jejunum; the release should be relatively fast, as orlistat should be available to exert their effect in duodenum and jejunum, and
iv) part G3 is designed to release of a part of the total dose of orlistat in duodenum and jejunum. Part b) and c) may be combined to part G2.

The composition suitable for use according to the invention may be a composition wherein G1 is in the form of inert cores coated with a composition comprising acarbose, G2A and G2B are combined to G2 and G2 is in the form of inert cores coated onto which acarbose and orlistat are applied and then provided with a coating with a protective polymer followed by coating with an enteric coating, and G3 is in the form of uncoated granules.

A protective polymer is typically present in a concentration of at least 10% w/w such as in a range of from 10-20% w/w, from 12 to 20% w/w, from 13 to 20% w/w, from 13.5 to 20% w/w based on the total weight of G2.

A composition for use according to the invention may comprises modified release granules, spheres or pellets comprising from 30 to 50% w/w of micronized orlistat, from 35 to 60% w/w of microcrystalline cellulose and from 10 to 18% w/w of polysorbate 80 based on the total weight of the modified release granules, spheres or pellets. In embodiments, part G3 comprises modified release granules, spheres or pellets comprising from 30 to 50% w/w of micronized orlistat, from 35 to 60% w/w of microcrystalline cellulose and from 10 to 18% w/w of polysorbate 80, based on the total weight of G3.

Typically, a composition for use according to the invention has
i) a concentration of acarbose in the first part G1 is in a range of from 25% w/w to about 50% w/w such as from about 30% w/w to about 45% w/w or about 40% w/w based on the total weight of part G1,
ii) a concentration of acarbose in the second part G2A or G2 is in a range of from about 0.5% w/w to about 4.5% w/w such as from about 1% w/w to about 4% w/w, from about 1.5% w/w to about 3.5% w/w, from about 2% w/w to about 3.5% w/w, from about 2.5% w/w to about 3.25% w/w or about 3% w/w based on the total weight of G2A or G2, whichever is relevant,
iii) a concentration of orlistat in part G2B or G2 is in a range of from 5% w/w to about 30% w/w such as from about 10% w/w to about 25% w/w, from about 10% w/w to about 20% w/w, from about 12% w/w to about 20% w/w or about 15.5% w/w based on the total weight of G2B or G2, whichever is relevant, and/or
iv) a concentration of orlistat in part G3 is in a range of from 20% w/w to about 50% w/w such as from about 25% w/w to about 50% w/w, from about 30% w/w to about 45% w/w, from about 35% w/w to about 45% w/w or about 40% w/w based on the total weight of G3.

As mentioned above, the composition may comprise modified release granules, spheres or pellets containing from 35 to 60% w/w of cellulose or a cellulose derivative such as microcrystalline cellulose based on the total weight of the modified release granules, spheres or pellets.

In a separate aspect, the invention relates to the use of a composition comprising orlistat and acarbose to improve quality of life in obese or overweight. All details and particulars mentioned and described for the aspects mentioned herein applies mutatis mutandis for this aspect.

### DETAILED DESCRIPTION

The present invention relates to a composition of orlistat and acarbose for use in medicine, wherein the composition is administered orally to obtain a synergistic effect with respect to weight loss and to obtain an improved quality of life compared with placebo, baseline or no treatment.

The clinical study reported herein resulted in the surprising result that use of a composition comprising orlistat and acarbose in the treatment of obesity in obese subjects lead to a marked improvement in the subject's quality of life. The clinical study used the well-recognized method RAND-36 for assessment of quality of life. RAND-36 is perhaps the most widely used health-related quality of life (HRQOL) survey instrument in the world today. It is comprised of 36 items that assess eight health concepts: physical functioning, role limitations caused by physical health problems, role limitations caused by emotional problems, social functioning, emotional well-being, energy/fatigue, pain, and general health perceptions. Physical and mental health summary scores are also derived from the eight RAND-36 scales. RAND-36 is a set of generic, coherent, and easily administered quality-of-life measures. These measures rely upon patient self-reporting and are now widely utilized by managed care organizations for routine monitoring and assessment of care outcomes in adult patients.

In the present context, overweight is defined as a BMI of 27 or more and obesity is defined as a BMI of 30 or more. In the present context, a subject having a BMI of 27 or more suffering from or being at risk of suffering from overweight-associated diseases (e.g., diabetes etc.) may need medical treatment to alleviate or prevent such diseases by reducing the body weight. However, it is contemplated that obese or overweight subjects with a BMI of 27 kg/m2 or more such as 29 kg/m2 or more or 30 kg/m2 or more will have a markedly improved quality of life when subject to a treatment for overweight/obesity involving administering a composition comprising orlistat and acarbose.

Typically, the improvement in quality of life is assessed by a questionnaire including questions to following domains: physical functioning, bodily pain, role limitations due to physical health problems, role limitations due to personal or emotional problems, emotional well-being, social functioning, energy/fatigue, and general health perception. As mentioned above, a suitable questionnaire is RAND-36 health survey.

In RAND-36 36 questions are addressed. It is possible to score answers to each question as such or score answers to the various domains. A mean overall health transition score can also be obtained. The answers given to the questions are compiled into eight dimensional points according to a standardized calculation procedure developed by the RAND corporation. This procedure enables comparisons over time and between populations. The procedure entails:
1) checking whether the entered values are reasonable (e.g., is between 1 and 5 if the response scale is 5-point). Numbers outside the allowed range are set as missing,
2) recoding raw data (incl. possible reversal of the response scale) to calculation values (e.g., 1->100),
3) calculating scale points for the eight RAND domains, and
4) giving an explanatory text (label) to the abbreviations of the domain names (Physical functioning, Role functioning/Physical etc).

As seen from the examples therein the improvement in mean overall health transition score is 10 points or more such as 12 points or more, 14 points or more, 15 points or more, 16 points or more compared with baseline.

If a placebo-controlled clinical study is performed, the comparison may or may in addition be with the placebo score.

In the present context, the term “baseline” indicates the situation just before treatment with a composition comprising orlistat and acarbose is initiated, i.e., in relation to quality of life, the questionnaire has been answered by a subject planned to undergo treatment with a composition comprising orlistat and acarbose, but just before start of treatment.

Typically, the improvement is in at least 5 of the following domains: physical functioning, bodily pain, role limitations due to physical health problems, role limitations due to personal or emotional problems, emotional well-being, social functioning, energy/fatigue, and general health perception.

Additionally, or alternatively, the improvement in mean score is increased by 10 or more compared to baseline.

As mentioned above, the Applicant has carried out clinical studies that showed a synergistic effect when acarbose and orlistat are administered orally and at the same time. The clinical study is described in detail in the experimental section herein. It is generally known that oral administration of orlistat for a period of 6 months results in a placebo adjusted reduction of body weight of approx. 2-3%. Of note, these studies are performed in a vetted population and together with lifestyle instructions Extrapolation from the literature it can be seen that oral administration of acarbose (using doses twice as high as in the current trial) for a time period of 6 months leads to a placebo adjusted reduction in body weight of approx. 0.5%. Accordingly, it would have been expected that oral administration of both acarbose and orlistat, in a non-vetted population, would give a placebo adjusted body weight reduction of approx. 2.5%. Surprisingly, from the clinical results reported herein an unexpected, marked reduction in body weight was seen. A placebo adjusted average relative body weight loss of more than 5% was observed at the clinical study.

In the present context, for therapeutic use, obesity is defined as a BMI of 30 or more. Overweight is defined as a BMI in a range of from 25 to less than 30.

Compared with placebo, the invention gives a body weight loss of at least 5%, such as at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700% or 800% or more. This percentage is calculated as: 100% * body weight loss in kg after treatment with a composition of the invention/body weight loss in kg after treatment with placebo (i.e. a composition without content of the active drug substances, acarbose and orlistat).

The synergistic effect reported in Example 2 was observed already after 13 weeks of treatment and the effect was further increased in the time period from 13 to 26 weeks of treatment. It is contemplated that the synergistic effect also is effective even at a longer treatment period, i.e. that the synergistic effect is obtained after 13 weeks or more such a after 14 weeks or more, after 15 weeks or more, after 16 weeks or more, after 17 weeks or more, after 18 weeks or more, after 19 weeks or more, after 20 weeks or more, after 21 weeks or more, after 22 weeks or more, after 23 weeks or more, after 24 weeks or more, after 25 weeks or more, or after 26 weeks or more of treatment.

The synergistic effect is obtained by oral administration of orlistat and acarbose in a weight ratio ranging from 2:1 to 4:1 such as a weight ratio of 3:1.

Orlistat and acarbose should be administered at the same time and preferably together with a meal. The administration may take place once daily, twice daily or three times daily. The daily dose of orlistat and acarbose depend on several individual factors such as the body weight of the subject to be treated, the risk-benefit profile relating to side effects compared with therapeutic effect etc. In general, the daily dose to an adult of orlistat is from 180 mg to 450 mg or more such as from 180 mg to 450 mg, from 270 mg to 450 mg, from 360 mg to 450 mg and the daily dose of acarbose is from 60 mg to about 150 mg or more such as from 90 mg to 150 mg.

A composition for use according to the invention may comprise 90 mg orlistat/30 mg acarbose, 120 mg orlistat/40 mg orlistat, 150 mg orlistat/50 mg acarbose, 180 mg orlistat/60 mg acarbose. It is typically administered orally three times daily.

The daily dose of orlistat for a child 5-10 years old weighing 40-60 kg is 120 mg, for a child 5-10 years old weighing 60-70 kg is 270 mg, and for a child older than 10 years old and/or weighing more than 70 kg is the same as for an adult.

The daily dose of acarbose for a child 5-10 years old weighing 40-60 kg is 60 mg, for a child 5-10 years old weighing 60-70 kg is 90 mg, and for a child older than 10 years old and/or weighing more than 70 kg is the same as for an adult.

For pediatric use an oral three times daily dose should be based on body weight; 40-60 kg: 60 mg orlistat/20 mg acarbose, 60-70 kg: 90 mg orlistat/30 mg acarbose, and above 70 kg: adult dose.

Typically, a composition of the invention comprises granules, spheres and/or pellets comprising orlistat and/or acarbose. The granules, spheres and/or pellets may be designed to release acarbose and/or orlistat in a modified manner. In the present context, the term “modified release” is intended to denote that the release of the active drug substance is manipulated by means of e.g. pharmaceutically acceptable excipients and/or coating materials; examples of coating materials that lead to a modified release are e.g. enteric coating materials;, which can be selected to release the active drug substance when pH is above a certain value such as e.g. pH above pH in the stomach; examples of pharmaceutically acceptable excipients that may lead to delayed release are e.g. celluloses or cellulose derivatives such as e.g. hydroxypropyl methylcellulose. Another way of obtaining a modified release may be by utilizing the water-soluble properties and/or pH-dependent solubility of the drug substances themselves.

As mentioned herein before the present invention is based on the Applicant's invention as described in WO 2016/097170. However, in order to achieve a release in vivo that is desired a modification of the composition has been necessary. Surprisingly, this modification has resulted in that a synergistic effect of the two drug substances has been obtained. This is a very important finding as it means that a faster and more efficient body weight loss can be obtained and moreover, a desired body weight loss can be obtained at a reduced period of time. A composition of the invention comprises granules, spheres or pellets. Some part of the composition is designed to avoid release of the active substances in the stomach (e.g., by coating of the granules, spheres or pellets, or by incorporating into the granules, spheres or pellets excipients that have pH-dependent release).

In WO 2016/097170 is described compositions comprising three or four different parts, wherein each part has a well-defined in vitro release pattern. However, the release rate from each part is based on simulations and in vitro investigations. Compositions for use according to the present invention comprise also three or four parts, G1, G2A, G2B and G3; if it only contains three parts, then G2A and G2B are part G2.

In the following is given a description focused on a combination product of orlistat and acarbose.

The release rates of the APIs are designed so that acarbose is released both in the stomach and some parts of the small intestine via defined different formulation principles, whereas orlistat is released throughout the small intestines, but at different rates, until the end of jejunum. By releasing the unchanged APIs at different rates, sufficient inhibition of digestive enzymes is achieved; enabling relevant amounts of undigested carbohydrates and lipids to reach the distal regions of the small intestine. The digested metabolites (fatty acids, monoacylglycerols and hexose) that is formed locally through local digestion will then act as ligands and stimulate the so-called gastro-intestinal brake effect.

In the present context the terms RR denotes rapid release, DR denotes delayed release and PR denotes prolonged release. The delayed release means that the release has been delayed, but when the release starts it may be rapid or prolonged. The subscripts DC denoted delayed coating, GASTRIC denotes that the release starts in the stomach, but there may still be release of the drug substance after passage into and through the small intestine until the end of jejunum, EC denotes an enteric coating, i.e. a coating with certain polymers that has a pH-cut off of about 4, i.e. they do not dissolve at acid pH and gradually begins to dissolve at about pH 4. Polymers may be employed having a pKₐ value of about 5.5, i.e., they begin to dissolve at about pH 5.5. Accordingly, as the drug substances are not released at pH below 4, PROX-SI denotes that the release should start and mainly take place in the proximal small intestine, and INTESTINAL denotes that the release should take place in the first part of small intestine until the end of jejunum.

This invention provides an oral pharmaceutical modified-release (MR) composition that is designed to
i) release a part of the total dose of acarbose in the stomach, but in a delayed manner in order to ensure that particles with acarbose will be well mixed with the food components and chyme in the postprandial stomach,
ii) release a part of the total dose of acarbose and a part of the total dose of orlistat in duodenum and jejunum; this release should be relatively fast, as both acarbose and orlistat should be available to exert their effect in duodenum and jejunum, and
iii) release of a part of the total dose of orlistat in duodenum and jejunum.

As mentioned above, various formulation principles can be used to prepare a composition for use according to the present invention. Such formulation principles can be seen from WO 2016/097170 to which reference is made. However, to obtain a synergistic effect, the inventors have developed a composition comprising acarbose and orlistat, wherein the composition contains individually distinct parts. The composition may contain three or four different parts:
a) a first part, G1, comprising from about 45% w/w to about 65% w/w such as from about 50% w/w to about 65% w/w, from about 55% w/w to about 65% w/w or about 60% w/w of the total dose of acarbose,
b) a second part, G2A, comprising from about 35% w/w to about 55% w/w such as from about 35% w/w to about 50% w/w, from about 35% w/w to about 45% w/w or about 40% w/w of the total dose of acarbose,
c) a third part, G2B, comprising from about 50% w/w to about 85% w/w such as from about 55% w/w to about 80% w/w, from about 60% w/w to about 80% w/w, from about 65% w/w to about 75% w/w, from about 68% w/w to about 75% w/w, from about 72% w/w to about 73% w/w such as about 72.2% w/w of the total dose of orlistat, and
d) a fourth part, G3, comprising from about 15 to about 50% w/w such as from about 20% w/w to 40% w/w, from about 25% to about 35% w/w, from about 25% to about 32% w/w, from about 27% w/w to about 28% w/w or about 27.8% w/w of the total dose of orlistat, and the total concentration of acarbose and orlistat, respectively, is 100% w/w;
if the composition only contains three parts, part b) and c) are combined. The combined part is called G2. The release patterns of the distinct parts are different as the individual parts are designed to release acarbose and orlistat in the different parts of the gastrointestinal tract.

Moreover, in order to obtain the desired release in vivo, the concentration of acarbose in the first part G1 is in a range of from 25% w/w to about 50% w/w such as from about 30% w/w to about 45% w/w or about 40% w/w based on the total weight of part G1. The concentration of acarbose in the second part G2A or G2 is in a range of from about 0.5% w/w to about 4.5% w/w such as from about 1% w/w to about 4% w/w, from about 1.5% w/w to about 3.5% w/w, from about 2% w/w to about 3.5% w/w, from about 2.5% w/w to about 3.25% w/w or about 3% w/w based on the total weight of G2A or G2, whichever is relevant. The concentration of orlistat in the second part G2B or G2 is in a range of from 5% w/w to about 30% w/w such as from about 10% w/w to about 25% w/w, from about 10% w/w to about 20% w/w, from about 12% w/w to about 20% w/w or about 15.5% w/w based on the total weight of G2B or G2, whichever is relevant. The concentration of orlistat in the third (or fourth part) G3 is in a range of from 20% w/w to about 50% w/w such as from about 25% w/w to about 50% w/w, from about 30% w/w to about 45% w/w, from about 35% w/w to about 45% w/w or about 40% w/w based on the total weight of G3.

In order to obtain a desired in vivo release of the active substances it is important to choose pharmaceutically acceptable excipients that control the release of the active substance. Especially the inventors addressed the release of orlistat from part(s), G2 (G2A, G2B) and G3 to obtain the desired release in vivo. The inventors found that the enteric polymer contained in G2 may have had a certain negative effect on the in vivo release of orlistat (and/or acarbose) from G2 (G2A, G2B). It turned out that the desired release in vivo could be obtained by minimizing direct contact between the drug substances and the enteric polymer. When G2 (G2A, G2B) is in the form of granules, spheres or pellets the direct contact between the drug substances and the enteric polymer can be minimized by coating the granules, spheres or pellets (before admixing or coating with an enteric polymer) with a protective layer. It has been found that the protective layer should have a certain thickness in order to ensure that the active substances in the G2 granule do not come into direct contact with the enteric polymer. The thickness is expressed as the concentration of protective layer in the final G2 (G2A, G2B) part and it should be in a concentration of at least 10% w/w such as in a range of from 10-20% w/w, from 12 to 20% w/w, from 13 to 20% w/w, from 13.5 to 20% w/w based on the weight of G2 (G2A, G2B).

Another observation was to obtain the right balance between the active substance and the pharmaceutically acceptable excipients imparting modified release properties to parts G2B or G2 and/or G3 to obtain the desired in vivo release. Furthermore, the in vivo release could be optimized by using orlistat in micronized form. Orlistat has a very poor water solubility (less than 0.001 g/ml) and using orlistat in micronized form increase the surface area and thereby enhances the rate of water solubility. Also, the use of a surfactant to ease the contact between orlistat and the fluid in the gastrointestinal tract had positive impact on the release rate in vivo.

The G3 part of the composition is intended to release orlistat in a delayed manner such that orlistat is effective in the proximal intestine. In order to achieve this, orlistat is used in micronized form and the concentration of orlistat in this part should be much smaller than originally envisaged in WO 2016/097170.

Accordingly, a composition according to the invention comprises a part G3, which comprises modified release granules, spheres or pellets comprising orlistat, wherein the modified release granules, spheres or pellets contains from 30 to 50% w/w of micronized orlistat based on the total weight of G3, the modified release granules, spheres or pellets comprising orlistat.

Moreover, the granules, spheres or pellets of G3 comprises from 35 to 60% w/w of cellulose or a cellulose derivative such as microcrystalline cellulose.

Accordingly, in an aspect of the invention a composition according to any one of the preceding claims comprising modified release granules, spheres or pellets comprising from 30 to 50% w/w of micronized orlistat, from 35 to 60% w/w of microcrystalline cellulose and from 10 to 18% w/w of polysorbate 80.

The present invention also provides compositions as described herein such a composition described above.

### Formulation of the G1, G2 and G3 Parts of the Composition

The G1 part of the composition is designed to release acarbose in a prolonged manner. The prolonged release is obtained by providing a G1 part that contains acarbose and a prolonged release polymer or a lipid. The prolonged release polymer typically has a poor water-solubility, i.e. it is a hydrophobic polymer, and may be selected from the group consisting of ethylcellulose, acrylates or acrylic acid derivatives, gelatin, coating agent selected from the group consisting of co-polymers based on polymethacrylic acid and methacrylates, ethyl acrylate and methyl acrylate, co-polymers of acrylic and methacrylic acid esters, hydroxypropyl methylcellulose phthalate, cellulose acetate phthalate, polyvinyl acetate phthalate or mixtures thereof. The lipid may be selected from fatty acids and/or esters, fatty alcohols, cetyl alcohol, stearyl alcohol, mineral oils, hydrogenated vegetable oils, vegetable oils, acetylated hydrogenated soybean oil glycerides, Castor oil, preferably solid at room temperature, most preferably hydrogenated vegetable oil.

The hydrophobic polymer or lipid is typically present in G1 in a concentration of from about 10% to about 50% w/w such as from about 15% to about 45% w/w, from about 20 to about 40% w/w, from about 15% to about 25% of the total weight of G1.

The hydrophobic polymer or lipid may be substituted by or supplemented with hydroxypropylmethylcellulose or a wax such as, e.g., glycerol monostearate, white wax, carnauba wax, stearyl alcohol, stearic acid, polyethylene glycol and triglycerides or mixtures thereof.

Hydroxypropylmethylcellulose or wax is typically present in G1 in a concentration of from about 3% w/w to about 50% w/w such as from about 3% w/w to about 45% w/w, from about 3% w/w to about 40% w/w, from about 3% to about 35% w/w, from about 3% to about 30% w/w, from about 3% to about 25% w/w, from about 4% w/w to about 20% w/w, from about 4% w/w to about 15% w/w, from about 4.5% w/w to about 10% w/w or from about 5% to about 9.5% w/w based on the total weight of G1. In some case, the concentration range is from about 10% to about 50% w/w such as from about 15% to about 45% w/w or from about 20 to about 40% w/w of the total weight of G1.

Moreover, in order to obtain the desired release in vivo, the concentration of acarbose in the first part G1 is in a range of from 25% w/w to about 50% w/w such as from about 30% w/w to about 45% w/w or about 40% w/w based on the total weight of part G1

As seen from the examples herein, G1 may be prepared based on a neutral core such as e.g., a microcrystalline cellulose core onto which a coating composition is applied containing acarbose.

The G2 part of the composition is designed to have a delayed release of acarbose and orlistat, but once release starts then it is relatively rapid. This release pattern is obtained by combining the drug substances with one or more surfactants (especially in order to increase the solubility of orlistat) and an enteric polymer, i.e., a polymer that has a pH dependent solubility such that it is not soluble at low pH (normally at pH 4 or less), but soluble at neutral/alkaline pH. The polymer may be incorporated into the formulation of G2, or it may be used as a coating material to coat the G2 formulation. As mentioned herein before, it is necessary to minimize any direct contact between the drug substances and the enteric polymer. This can be obtained by providing spheres, granules of pellets containing the active drug substances with a protective coating layer. Suitable polymers for use as protective polymers include cellulose or cellulose derivatives such as hydroxypropyl methylcellulose or other film-forming polymers.

Moreover, orlistat must be used in micronized form in order to achieve a desired release in vivo. Accordingly, average particle size of orlistat should be 50 microns or below such as 20 microns or below, 10 microns or below or 5 microns or below.

The surfactant is typically selected from the group consisting of anionic, cationic or non-ionic surfactant. Non-ionic are e.g., polysorbate 20, polysorbate 21, polysorbate 40, polysorbate 60, polysorbate 61, polysorbate 65, polysorbate 80, polysorbate 81, polysorbate 85, polysorbate 120, sorbitan monoisostearate, sorbitan monolaurate, sorbitan monopalmitate, sorbitan monostearate, sorbitan monooleate, sorbitan sesquioleate, sorbitan trioleate, glyceryl monooleate and polyvinylalcohol. Anionic surfactants include docusate sodium and sodium lauryl sulphate. Cationic surfactants include e.g., benzalkonium chloride, benzethonium chloride and cetrimide.

The total concentration of surfactants is typically present in G2 in a concentration of from about 0.5% to about 30% w/w of the total weight of G2. Preferably, the concentration is from about 1% w/w to about 10% w/w such as from about 1% w/w to about 8% w/w, from about 1% w/w to about 5% w/w based on the total weight of G2.

The enteric polymer may also be a coating agent selected from the group consisting of co-polymers based on polymethacrylic acid and methacrylates, ethyl acrylate and methyl acrylate, co-polymers of acrylic and methacrylic acid esters, hydroxypropyl methylcellulose phthalate, hydroxypropyl methylcellulose acetate succinate, cellulose acetate phthalate, polyvinyl acetate phthalate or mixtures thereof, such as that which is commercially available from Shin-Etsu and Seppic under the name Aqoat® AS-LG (hypromellose acetate succinate).

The enteric polymer is typically an acrylate or acrylic acid polymer or co-polymer. The acrylic polymer may comprise one or more ammonio methacrylate copolymers. Ammonio methacrylate copolymers are well known in the art and are described in NF XVII as fully polymerized copolymers of acrylic and methacrylic acid esters with a low content of quaternary ammonium groups.

The acrylic polymer may be used in the form of an acrylic resin lacquer used in the form of an aqueous dispersion, such as that which is commercially available from Rohm Pharma under the tradename Eudragit® or from Colorcon under the tradename Acryl-EZE®. The acrylic coating may comprise a mixture of two acrylic resin lacquers commercially available from Evonik under the tradenames Eudragit® RL 30 D and Eudragit® RS 30 D, respectively. Eudragit® RL 30 D and Eudragit® RS 30 D are copolymers of acrylic and methacrylic esters with a low content of quaternary ammonium groups, the molar ratio of ammonium groups to the remaining neutral (meth)acrylic esters being 1:20 in Eudragit® RL30 D and 1:40 in Eudragit® RS 30 D.

Eudragit® RL/RS mixtures are insoluble in water and in digestive fluids. However, coatings formed from the same are swellable and permeable in aqueous solutions and digestive fluids. The Eudragit® RL/RS dispersions may be mixed together in any desired ratio in order to ultimately obtain a modified release formulation having a desirable dissolution profile.

In the G2 part, the enteric polymer is typically present in a concentration of from about 15 to about 50% w/w based on the total weight of the G2 formulation. It is preferred that the concentration is from about 20% w/w to about 40% w/w such as from about 15% w/w to about 40% w/w, from about 15% w/w to about 35% w/w, from about 15% w/w to about 30% w/w, from about 20 to about 25% w/w based on the total weight of G2.

The concentration of the protective polymer in G2 (G2A, G2B) part should be at least 10% w/w such as in a range of from 10-20% w/w, from 12 to 20% w/w, from 13 to 20% w/w, from 13.5 to 20% w/w based on the total weight of G2 (G2A, G2B).

The concentration of acarbose in the second part G2A or G2 is in a range of from about 0.5% w/w to about 4.5% w/w such as from about 1% w/w to about 4% w/w, from about 1.5% w/w to about 3.5% w/w, from about 2% w/w to about 3.5% w/w, from about 2.5% w/w to about 3.25% w/w or about 3% w/w based on the total weight of G2A or G2, whichever is relevant. The concentration of orlistat in the second part G2B or G2 is in a range of from 5% w/w to about 30% w/w such as from about 10% w/w to about 25% w/w, from about 10% w/w to about 20% w/w, from about 12% w/w to about 20% w/w or about 15.5% w/w based on the total weight of G2B or G2, whichever is relevant.

The G3 part is designed to release orlistat in a prolonged manner. Orlistat may be release at a low degree already in the stomach. Orlistat is very poor water-soluble and in order to achieve the desired release, orlistat is combined with one or more surfactants. The surfactant may be one or more of those mentioned above under G2. The surfactant is present in G3 in a concentration from about 1% to about 30% w/w of the total weight of the G3 formulation. Preferably, it is present from about 2% to about 20% w/w, from about 3% to about 20% w/w from about 5% w/w to about 20% w/w, from about 10% w/w to about 15% w/w.

Moreover, orlistat must be used in micronized form in order to achieve a desired release in vivo. Accordingly, average particle size of orlistat should be 50 microns or below such as 20 microns or below, 10 microns or below or 5 microns or below.

Alternatively, or additionally, the release of orlistat from G3 can be obtained by incorporation of a water-soluble or water-swellable polymer such as hydroxypropylmethylcellulose or other cellulose derivatives like e.g., methylcellulose, carboxymethylcellulose, hydroxypropylcellulose, micro-crystalline cellulose or the like.

Such a water-soluble polymer is typically incorporated into the G3 formulation in a concentration of from about 35 to about 60% w/w such as from about 35% w/w to about 55% w/w, from about 35% to about 50%, from about 40% w/w to about 50% w/w based on the total weight of G3. In some case, the concentration may be from about 70 to about 90% w/w based on the total weight of G3. Preferably, the concentration is from about 40 to about 50% w/w.

The concentration of orlistat in the third (or fourth part) G3 is in a range of from 20% w/w to about 50% w/w such as from about 25% w/w to about 50% w/w, from about 30% w/w to about 45% w/w, from about 35% w/w to about 45% w/w or about 40% w/w based on the total weight of G3.

The G1, G2 (or G2A, G2B) and G3 parts may also contain other pharmaceutically acceptable ingredients selected from those mentioned herein. Moreover, in order to manufacture a final composition G1, G2 (or G2A, G2B), and/or G3 may be admixed with one or more pharmaceutically acceptable excipient or G1, G2 (or G2A, G2B), and/or G3 may be coated e.g., with a film coating or with a coating that hinders or reduces negative impact of one part to another part.

The part G1 of the composition may be in the form of granules, spheres, pellets, minitablets etc. or part G1 is incorporated into a two-layer tablet, where part G1 is contained in one of the two layers. The layer containing part G1 may be provided with a delayed release coating.

Part G2, or G2A and G2B, of the composition may be in the form of granules, spheres, pellets, minitablets etc. containing an enteric polymer or provided with an enteric coating, or G2, or G2A and G2B, is incorporated into a two-layer tablet, where part G2, or G2A and G2B, is contained in one of the two layers and the layer containing part G2, or G2A and G2B, is provided with an enteric coating.

Part G3 may be in the form of granules, spheres, pellets, minitablets etc. or it is contained in a two-layer tablet, wherein part G3 is contained in one of the two layers.

The final modified-release composition according to the invention may be in the form of a multiple-unit tablet, a bi-layer multiple-unit tablet, a coated tablet, a multiple-unit capsule or a multiple-unit oral powder. Typically, G1, G2, or G2A and G2B, and G3 are in the form of pellets, granules, spheres or the like, and the modified-release composition according to the invention is in the form of a multiple-unit tablet, capsule, sachet or powder.

In a preferred aspect, G1 is in the form of inert cores coated with a coating composition comprising acarbose; G2 is in the form of inert cores coated onto which the drug substances are applied and then provided with a protective coating followed by coating with an enteric coating. G3 is in the form of uncoated granules.

Other pharmaceutically acceptable excipients may be included in the G1, G2 or G3 formulations.

However, it is to be understood that a synergistic effect also should be obtained administering two compositions, one containing acarbose and one containing orlistat and where the acarbose composition comprises parts G1 and G2A and the orlistat composition comprises parts G2B and G3. To obtain a synergistic effect, the compositions should be administered to the subject at the same time.

A composition according to the present invention comprises parts G1, G2 (alternatively G2A and G2B) and G3 and, optionally, one or more pharmaceutically acceptable excipients. Such a composition comprising from 30 to 50% w/w of micronized orlistat, from 35 to 60% w/w of microcrystalline cellulose and from 10 to 18% w/w of polysorbate 80 based on the total weight of the composition.

### Compositions of the Invention

The present invention also provides a composition comprising two or more parts with different release pattern. The parts are denoted G2 or G2B and G3. Such a composition contains
i) a part G2B, comprising from about 50% w/w to about 85% w/w such as from about 55% w/w to about 80% w/w, from about 60% w/w to about 80% w/w, from about 65% w/w to about 75% w/w, from about 68% w/w to about 75% w/w, from about 72% w/w to about 73% w/w such as about 72.2% w/w of the total dose of orlistat, and
ii) a part, G3, comprising from about 15 to about 50% w/w such as from about 20% w/w to 40% w/w, from about 25% to about 35% w/w, from about 25% to about 32% w/w, from about 27% w/w to about 28% w/w or about 27.8% w/w of the total dose of orlistat, and the total concentration of acarbose and orlistat, respectively, is 100% w/w,
wherein orlistat is present in the parts in the form of micronized orlistat.

More specifically, the concentration of orlistat in the part G2B is in a range of from 5% w/w to about 30% w/w such as from about 10% w/w to about 25% w/w, from about 10% w/w to about 20% w/w, from about 12% w/w to about 20% w/w or about 15.5% w/w based on the total weight of G2B. The concentration of orlistat in the part G3 is in a range of from 20% w/w to about 50% w/w such as from about 25% w/w to about 50% w/w, from about 30% w/w to about 45% w/w, from about 35% w/w to about 45% w/w or about 40% w/w based on the total weight of G3.

The composition may further contain a part G1 and G2A as described herein and part G2A and G2B may be mixed for form part G2.

All details given herein with respect to parts G1, G2/G2A/G2B) and G3 apply mutatis mutandis for the above-mentioned compositions.

The invention also provides a composition containing three or four different parts:
a) a first part, G1, comprising from about 45% w/w to about 65% w/w such as from about 50% w/w to about 65% w/w, from about 55% w/w to about 65% w/w or about 60% w/w of the total dose of acarbose,
b) a second part, G2A, comprising from about 35% w/w to about 55% w/w such as from about 35% w/w to about 50% w/w, from about 35% w/w to about 45% w/w or about 40% w/w of the total dose of acarbose,
c) a third part, G2B, comprising from about 50% w/w to about 85% w/w such as from about 55% w/w to about 80% w/w, from about 60% w/w to about 80% w/w, from about 65% w/w to about 75% w/w, from about 68% w/w to about 75% w/w, from about 72% w/w to about 73% w/w such as about 72.2% w/w of the total dose of orlistat, and
d) a fourth part, G3, comprising from about 15 to about 50% w/w such as from about 20% w/w to 40% w/w, from about 25% to about 35% w/w, from about 25% to about 32% w/w, from about 27% w/w to about 28% w/w or about 27.8% w/w of the total dose of orlistat, and the total concentration of acarbose and orlistat, respectively, is 100% w/w;
if the composition only contains three parts, part b) and c) are combined. The combined part is called G2. The release patterns of the distinct parts are different as the individual parts are designed to release acarbose and orlistat in the different parts of the gastrointestinal tract.

Moreover, in order to obtain the desired release in vivo, the concentration of acarbose in the first part G1 is in a range of from 25% w/w to about 50% w/w such as from about 30% w/w to about 45% w/w or about 40% w/w based on the total weight of part G1. The concentration of acarbose in the second part G2A or G2 is in a range of from about 0.5% w/w to about 4.5% w/w such as from about 1% w/w to about 4% w/w, from about 1.5% w/w to about 3.5% w/w, from about 2% w/w to about 3.5% w/w, from about 2.5% w/w to about 3.25% w/w or about 3% w/w based on the total weight of G2A or G2, whichever is relevant. The concentration of orlistat in the second part G2B or G2 is in a range of from 5% w/w to about 30% w/w such as from about 10% w/w to about 25% w/w, from about 10% w/w to about 20% w/w, from about 12% w/w to about 20% w/w or about 15.5% w/w based on the total weight of G2B or G2, whichever is relevant. The concentration of orlistat in the third (or fourth part) G3 is in a range of from 20% w/w to about 50% w/w such as from about 25% w/w to about 50% w/w, from about 30% w/w to about 45% w/w, from about 35% w/w to about 45% w/w or about 40% w/w based on the total weight of G3.

All details given herein with respect to parts G1, G2/G2A/G2B) and G3 apply mutatis mutandis for the above-mentioned compositions.

### Orlistat (Tetrahydrolipstatin)

### Chemical structure of orlistat ((S)-((S)-1-((2S,3S)-3-Hexyl-4-oxooxetan-2-yl)tridecan-2-yl) 2-formamido-4-methylpentanoate)

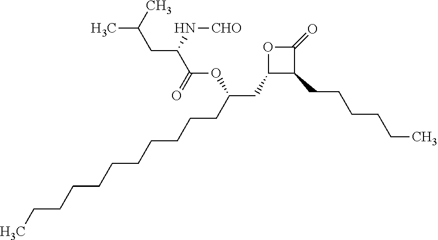 [SMILES: CCCCCCCCCCC[C@@H](C[C@@H]1OC(=O)[C@H]1CCCCCC)OC(=O)[C@H](CC(C)C)NC=O]

Orlistat may be prepared from biological material (Streptomyces toxytricin) or it may be prepared synthetically or semi-synthetically.

According to the literature, orlistat appears in two different crystal forms, Form I and Form II. The melting point of Form I and Form Il is 44° C. and 43° C. respectively. The product marketed by Roche under the name Xenical® capsules in Sweden contains Form II. No salt forms of orlistat seem to exist. It is practically insoluble in water.

In the present context the term “orlistat” covers the above-mentioned chemical structure as well as any optical isomer thereof as well as any crystal form, any polymorph, any hydrate, any pharmaceutically acceptable or any prodrug thereof.

Orlistat is a local inhibitor of gastric and pancreatic lipases in the GI tract and acts by preventing intestinal absorption of dietary fats through inhibition of luminal digestion. The physicochemical condition in the stomach and along the small intestine is very dynamic and this activity and the inhibition kinetics of orlistat will differ significantly. These dynamic GI conditions are considered in the designs of this fixed oral MR dosage form. The fraction of the oral orlistat dose absorbed from a conventional dosage form (Xenical®) is low (<3%) and accordingly the plasma exposure is low (<5 ng/ml). However, orlistat, although safe, is associated with side-effects that severely hamper compliance. In clinical trials, about 25% or more of the patients complain about GI side-effects including diarrhoea, oily spotting and faecal urgency. This, in conjunction with the rather modest effect on weight, makes Xenical® less attractive for the vast majority of obese patients. However, in a report FDA clearly stated that Xenical® is safe and has clinical benefit. Clinical use of orlistat in an oral modified-release (MR) dosage form does not only decreases fat GI absorption by preventing triglycerides from being broken down to free fatty acids and monoacylglycerols; orlistat also changes GI transit time and affects satiety through many of the cell types mentioned above and below.

In the stomach, the reduced lipid digestion caused by orlistat increases gastric emptying (food is delivered faster to the duodenum). If the meal is high in fat, diarrhoea might occur within 30 min from meal initiation. This diarrhoea is most probably due to the fact that food in the stomach normally triggers emptying of the colon. This signal, in combination of supra-normal amounts of fat in the faeces from previous meals (which leads to less water absorbed during colon transit), may cause the diarrhoea. Possibly, high fat meals will further augment the stomach-to-colon signal, thereby aggravating the situation. As fatty acids, and not intact triglycerides, are the ligands for the receptors in the GI tract, many of the above-mentioned hormones will be secreted at a lower level when the digestion of lipids normally occurring in the stomach is inhibited. Of note:
1) In the duodenum, the fatty acid signal to CCK will be weaker, and less bile will be secreted, which further decreases fat digestion.
2) The normal meal induced decrease of appetite stimulating hormone ghrelin will be attenuated.
3) The L-cells (which secretes the incretins) will also secrete less GLP-1, leading to a smaller ileal brake.

The undigested triglycerides will enter the colon, and as mentioned above, fat only enters colon in small amounts. Larger amounts of fat will lead to faster propulsion through the colon and less water will be absorbed. In summary, the current way of delivering orlistat in conventional dosage form (that includes drug release in the stomach) to the GI tract on the one hand removes calories in the form of intact undigested triglycerides, but on the other hand causes a lot of side effects and bypasses many of the appetite adjusting systems in the GI tract and also increases gastric emptying rate which in fact reduces the feeling of fullness and increases appetite.

### Acarbose

### Chemical structure of acarbose (O-4,6-Dideoxy-4-[[(1S,4R,5S,6S)-4,5,6-trihydroxy-3-(hydroxymethyl)cyclohex-2-enyl]amino]-α-D-glucopyranosyl-(1→4)-O-α-D-gluco-pyranosyl-(1→4)-D-glucopyranose

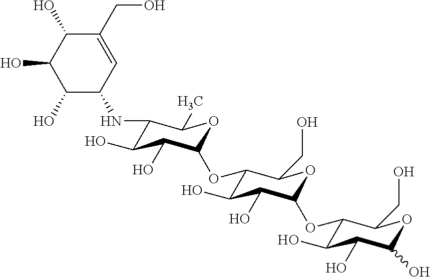

Acarbose may be prepared from biological material (Actinoplanes) or it may be prepared synthetically or semi-synthetically.

No information about the crystal form of acarbose could be found in the literature. However, some sources indicate that acarbose may be amorphous and no salt forms of acarbose seems to exist. According to Ph. Eur. it is very soluble in water.

In the present context the term “acarbose” covers the above-mentioned structure as well as any optical isomer thereof as well as any crystal form, any polymorph, any hydrate, any pharmaceutically acceptable or any prodrug thereof.

Acarbose (Glucobay®) is a competitive α-glucosidase and pancreatic α-amylase inhibitor, which inhibits the hydrolysis of oligosaccharides during GI luminal digestion of a meal. Acarbose is currently used as a diabetic drug, mainly in Asia, but only scarcely in Western countries. By inhibiting the luminal digestion and subsequent absorption of carbohydrates, the concentrations of glucose in blood sugar increases slower postprandially, and the patient's insulin need is reduced. The low intestinal permeability of acarbose (due to its hydrophilic properties) leads to less than 5% of the drug being absorbed after oral administration. The low GI absorption and bioavailability results in very low plasma exposure, which makes acarbose considered as a safe drug without systemic side-effects. As with orlistat, a large part of the patients using acarbose reports GI tolerability problems (mainly flatulence, diarrhoea as well as GI and abdominal pains), which limits its current clinical use in Western countries. The magnitude of GI side effects is directly associated with the strength of the oral dose, in a stepwise manner. Furthermore, the acarbose side effects seem also to be “diet driven”. The higher consumption of carbohydrates, and perhaps slightly more “resistant” carbohydrates (with a slightly slower digestion), in Asian countries seems to reduce the side-effect rate. Moreover, patients are recommended to slowly introduce acarbose by using 50 mg per day during 1-3 weeks time and then slowly increase the oral dose up to 100 mg per meal. As more undigested carbohydrates reach further down in the GI tract, more enzymes are being produced locally in the distal small intestine to deal with the undigested carbohydrates. Although acarbose also removes ligands from various cell types throughout the GI tract, some noteworthy differences are observed. Acarbose will reduce gastric emptying rate, possibly by delivering less ligands to GIP secreting K-cells in the proximal small intestine, and more ligands to distal GLP-1 secreting L-cells. Acarbose will also cause more undigested polysaccharides to enter the proximal colon, where bacteria will ferment the polysaccharides, and the resulting short chain fatty acids can bind to L-cells and augment the ileal brake.

It should be understood that any feature and/or aspect discussed above in connections with the compositions apply by analogy to the methods described herein.

The following figure examples are provided below to illustrate the present invention. They are intended to be illustrative and are not to be construed as limiting in any way.

### BRIEF DESCRIPTION OF THE FIGURES

FIG. 1 shows details regarding the clinical study described in Example 2 herein and the results thereof.

FIG. 2 shows relative weight loss during the 26-week study period, see Example 2.

FIG. 3 shows the proportion of participants losing more than 5% and 10% weight. More patients in the active treatment groups had lost 5% or more (p<0.0001 for both active treatment vs placebo) and 10% or more (p<0.1 for both active treatment groups vs placebo) at week 26.

FIG. 4 shows the correlation between relative weight loss and change in the RAND-36 item health transition (from baseline to week 26) in the three arms.

### EXAMPLES

### Example 1—A Composition Comprising Three Parts, G1, G2 and G3 was Prepared

| | |
|---|---|
| | EMP16-02 |
| API | Quantity per |
| (% wt.) | capsule (g) |

| Component | EMP16-02 | 60/20 | 90/30 |
|---|---|---|---|
| G1 | Acarbose | 0.03 | 0.04 |
| | 39.2 | | |
| G2 | Orlistat/Acarbose | 0.28 | 0.41 |
| | 15.6/3.1 | | |
| G3 | Orlistat | 0.04 | 0.06 |
| | 40.0 | | |

Capsule excipients

| | | | |
|---|---|---|---|
| Magnesium stearate, | Lubricant | 0.002 | 0.003 |
| vegetable | | | |
| Hard gelatin capsule | Dosage unit | 0.10 | 0.12 |
| white/white size 00 | container | | |
| Total weight | | 0.45 | 0.64 |

The composition of EMP16-02-60/20 modified-release capsules containing orlistat 60 mg/unit and acarbose 20 mg/unit is described below:

| Material | Function | mg/unit | w/W % | Standard |
|---|---|---|---|---|
| Orlistat, micronized | Active ingredient | 60.0 | 17.1 | Current USP/NF |
| Acarbose | Active ingredient | 20.0 | 5.7 | Current EP |
| Microcrystalline cellulose | Neutral core | 66.4 | 18.9 | Current USP/NF |
| (Celphere CP 203, 150-300 μm) | | | | |
| Microcrystalline cellulose, PH-101 | Filler/Binder | 19.2 | 5.5 | Current EP |
| Ethylcellulose | Excipient in | 6.7 | 1.9 | Current USP/NF |
| (Surelease Ethylcellulose Dispersion | diffusion coating | | | |
| Aqueous type B) | | | | |
| Hydroxypropyl cellulose | Binder | 43.2 | 12.3 | Current USP/NF |
| (Klucel EF Pharm) | | | | |
| Hypromellose | Binder | 0.9 | 0.2 | Current EP |
| (Pharmacoat 606) | | | | |
| Sepifilm LP 914 | Seal-coat | 38.9 | 11.1 | Supplier |
| | | | | monograph |
| Hypromellose | Excipient in | 0.7 | 0.2 | Current EP |
| (Methocel E3 Premium LV) | diffusion coating | | | |
| Polysorbate 80 | Surfactant | 9.8 | 2.8 | Current EP |
| Ammoniac | Polymer | 1.6 | 0.4 | Current EP |
| | neutralisation | | | |
| Hypromellose acetate succinate | Polymer for gastro | 62.9 | 17.9 | Current USP/NF |
| (Aqoat AS-LG) | resistance | | | |
| Talc | Anti-tacking agent | 18.9 | 5.4 | Current EP |
| Magnesium stearate, vegetable | Lubricant | 1.5 | 0.4 | Current USP/NF |
| — | Sum | 350.7 | 100 | — |
| Hard gelatin capsule white/white | Dosage unit | 95.0 | — | Supplier |
| size 0 | container | | | monograph and |
| | | | | Current EP |
| — | Total | 445.7 | — | — |

The composition of EMP16-02-90/30 modified-release capsules containing orlistat 90 mg/capsule and acarbose 30 mg/unit is described below:

| Material | Function | mg/unit | w/w % | Standard |
|---|---|---|---|---|
| Orlistat, micronized | Active ingredient | 90.0 | 17.1 | Current USP/NF |
| Acarbose | Active ingredient | 30.0 | 5.7 | Current EP |
| Microcrystalline cellulose | Neutral core | 99.8 | 19.0 | Current USP/NF |
| (Celphere CP 203, 150-300 μm) | | | | |
| Microcrystalline cellulose, PH-101 | Filler/Binder | 28.8 | 5.5 | Current EP |
| Ethylcellulose | Excipient in | 10.1 | 1.9 | Current USP/NF |
| (Surelease Ethylcellulose Dispersion | diffusion coating | | | |
| Aqueous type B) | | | | |
| Hydroxypropyl cellulose | Binder | 64.9 | 12.3 | Current USP/NF |
| (Klucel EF Pharm) | | | | |
| Hypromellose | Binder | 1.3 | 0.2 | Current EP |
| (Pharmacoat 606) | | | | |
| Sepifilm LP 914 | Seal-coat | 58.4 | 11.1 | Supplier |
| | | | | monograph |
| Hypromellose | Excipient in | 1.1 | 0.2 | Current EP |
| (Methocel E3 Premium LV) | diffusion coating | | | |
| Polysorbate 80 | Surfactant | 14.6 | 2.8 | Current EP |
| Ammoniac | Polymer | 2.3 | 0.4 | Current EP |
| | neutralisation | | | |
| Hypromellose acetate succinate | Polymer for gastro | 94.5 | 17.9 | Current USP/NF |
| (Aqoat AS-LG) | resistance | | | |
| Talc | Anti-tacking agent | 28.3 | 5.4 | Current EP |
| Magnesium stearate, vegetable | Lubricant | 2.3 | 0.4 | Current USP/NF |
| — | Sum | 526.4 | 100 | — |
| Hard gelatin capsule white/white | Dosage unit | 118.0 | — | Supplier |
| size 00 | container | | | monograph and |
| | | | | Current EP |
| — | Total | 644.4 | — | — |

The composition of G1 granules containing acarbose 391.5 mg/g is described below:

| Material | Function | mg/g | w/w % | Standard |
|---|---|---|---|---|
| Acarbose | Active ingredient | 391.5 | 39.2 | Current EP |
| Microcrystalline cellulose | Neutral core | 329.0 | 32.9 | Current |
| (Celphere CP 203, 150-300 μm) | | | | USP/NF |
| Hypromellose | Binder | 29.5 | 2.9 | Current EP |
| (Pharmacoat 606) | | | | |
| Hypromellose | Excipient in | 25.0 | 2.5 | Current EP |
| (Methocel E3 Premium LV) | diffusion coating | | | |
| Ethylcellulose | Excipient in | 225.0 | 22.5 | Current |
| (Surelease Ethylcellulose Dispersion | diffusion coating | | | USP/NF |
| Aqueous type B) | | | | |
| Granulation liquid | Solvent | —*⁾ | q.s.*⁾ | Current EP |
| — | Sum | 1000 | 100 | — |

*⁾Evaporates during manufacturing process

The composition of G2 granules containing orlistat 155.8 mg/g and acarbose 29.7 mg/g is described below:

| Material | Function | mg/g | w/w % | Standard |
|---|---|---|---|---|
| Orlistat, micronized | Active ingredient | 155.8 | 15.56 | Current USP/NF |
| Acarbose | Active ingredient | 29.7 | 2.97 | Current EP |
| Microcrystalline cellulose | Neutral core | 204 | 20.4 | Current USP/NF |
| (Celphere CP 203, 150-300 μm) | | | | |
| Polysorbate 80 | Surfactant | 14.1 | 1.41 | Current EP |
| Hydroxypropyl cellulose | Binder | 155.8 | 15.58 | Current USP/NF |
| (Klucel EF Pharm) | | | | |
| Sepifilm LP 914 | Seal-coat | 140.2 | 14.02 | Supplier |
| | | | | Monograph |
| Hypromellose acetate succinate | Polymer for gastro- | 226.8 | 22.68 | Current USP/NF |
| (Aqoat AS-LG) | resistance | | | |
| Ammoniac | Polymer | 5.6 | 0.56 | Current EP |
| | neutralisation | | | |
| Talc | Anti-tacking agent | 68 | 6.8 | Current EP |
| Granulation liquid | Solvent | —*⁾ | q.s.*⁾ | Current EP |
| — | Sum | 1000 | 100 | |

*⁾Evaporates during manufacturing process

The composition of G3 granules containing 400.0 mg/g is described below:

| Material | Function | mg/g | w/w % | Standard |
|---|---|---|---|---|
| Orlistat, micronized | Active ingredient | 400.0 | 40.0 | Current USP/NF |
| Microcrystalline cellulose, PH-101 | Filler/binder | 460.0 | 46.0 | Current EP |
| Polysorbate 80 | Surfactant | 140.0 | 14.0 | Current EP |
| Granulation liquid | Solvent | —*⁾ | q.s*⁾ | Current EP |
| — | Sum | 1000 | 100 | — |

*⁾Evaporates during manufacturing process

### Example 2—Clinical Study

A multi-centre, double-blind, placebo-controlled, randomized study in overweight and obese patients was conducted during twenty-six weeks.

The primary objective was to evaluate the effect of EMP16-02 (120 mg orlistat/40 mg acarbose and 150 mg orlistat/50 mg acarbose) on relative body weight loss after a 26-week period of oral treatment as compared to placebo.

The secondary objectives were:
i) To assess the effect of two different doses of EMP16-02 (120 mg O/40 mg A and 150 mg O/50 mg A) on relative and absolute body weight loss during a 26-week period of oral treatment as compared to placebo;
ii) To assess the effect of two different doses of EMP16-02 (120 mg O/40 mg A and 150 mg O/50 mg A) on other anthropometric characteristics during a 26-week period of oral treatment as compared to placebo;
iii) To assess the effect of two different doses of EMP16-02 (120 mg O/40 mg A and 150 mg O/50 mg A) on satiety and meal pattern during a 26-week period of oral treatment as compared to placebo;
iv) To assess the effect of two different doses of EMP16-02 (120 mg O/40 mg A and 150 mg O/50 mg A) on fasting insulin, glucose metabolism markers, lipid metabolism markers and inflammation markers during a 26-week period of oral treatment as compared to placebo;
v) To assess the effect of two different doses of EMP16-02 (120 mg O/40 mg A and 150 mg O/50 mg A) on blood pressure during a 26-week period of oral treatment as compared to placebo;
vi) To assess the effect of two different doses of EMP16-02 (120 mg O/40 mg A and 150 mg O/50 mg A) on quality of life during a 26-week period of oral treatment as compared to placebo;
vii) To assess the relationship between drop-out(s) and tolerability for two different doses of EMP16-02 (120 mg O/40 mg A and 150 mg O/50 mg A) during a 26-week period of oral treatment as compared to placebo;
viii) To assess the safety and gastrointestinal (GI) tolerability of two different doses of EMP16-02 (120 mg O/40 mg A and 150 mg O/50 mg A) during a 26-week period of oral treatment as compared to placebo.

### Exploratory Objectives:

To assess the effect of 2 different doses of EMP16-02 (120 mg O/40 mg A and 150 mg O/50 mg A) on fasting plasma/serum levels of apolipoprotein A1 (ApoA1) and apolipoprotein B (ApoB) during a 26-week period of oral treatment as compared to placebo.
To assess the pre-dose plasma level of orlistat and acarbose at steady state.
To evaluate the effect of 2 different doses of EMP16-02 (120 mg O/40 mg A and 150 mg O/50 mg A) on relative and absolute body weight loss 6 months after completion of a 26-week period of oral treatment as compared to placebo.
To evaluate the effect of 2 different doses of EMP16-02 (120 mg O/40 mg A and 150 mg O/50 mg A) on HbA1c concentration 6 months after completion of a 26-week period of oral treatment as compared to placebo.
To evaluate the effect of 2 different doses of EMP16-02 (120 mg O/40 mg A and 150 mg O/50 mg A) on blood pressure 6 months after completion of a 26-week period of oral treatment as compared to placebo.

### Diagnosis and Main Eligibility Criteria

Male and female patients with overweight or obesity, defined as body mass index (BMI) ≥_30, or _≥28_kg/m² in the presence of other risk factors (e.g., hypertension, glucose dysregulation such as impaired glucose tolerance and type 2 diabetes mellitus (T2DM), and/or dyslipidemia).
Aged ≥18_and_≤75 years._
Willing and able to give written informed consent for participation in the study.
Body weight stable (<5% reported change during the 3 months preceding screening and randomization).

The primary endpoint was the relative % change from baseline in body weight after 26 weeks of treatment with EMP16-02 (120 mg O/40 mg A) as compared to placebo.

A total of 156 patients were enrolled in the study.

The patients were randomised to either of two doses of EMP16-02:
1. EMP16-02 120 mg O/40 mg A
2. EMP16-02 150 mg O/50 mg A
3. Placebo (identical capsules)

For EMP16-02 120/40, 2 capsules of EMP16-02 60/20 are used.

For EMP16-02 150/50, 1 capsule of EMP16-02 60/20 and 1 capsule of EMP16-02 90/30 are used.

Blood sampling (fasting), and anthropometric measurements were performed. Patients received electronic diary instructions and were be asked to fill in a satiety and craving questionnaire before breakfast (at the clinic), and then once every hour for 4 hours until before lunch (at home). A standardised breakfast was served at the clinic. Halfway through breakfast at Visit 2, all patients received a placebo capsule independent of the treatment arm to which the patients had been randomised, to provide patients with the opportunity to train on self-administering the IMP under supervision of clinic staff. The patients also received instructions for filling in more questionnaires regarding health and quality of life, meal pattern, activity and sleep, and gastrointestinal symptoms (gastrointestinal rating scale [GSRS]).

The patients were instructed to take EMP16-02 or placebo halfway through each meal, together with approximately 100-200 ml water (or other drink) on all subsequent treatment days. Once IMP had been handed out, the patients were free to leave the clinic. The first randomised IMP dose were taken during lunch (or the next meal) at home.

Patients randomised to EMP16-02 started with a run-in period of 6 weeks during which the dose was sequentially increased. From week 7, all patients reached their final intended dose and a 20-week treatment and observation period started. The run-in phase started at a dose of 60 mg O and 20 mg A TID, which sequentially was increased with 30 mg O/10 mg A every two weeks until the target doses of 120 mg O/40 mg A TID (for the lower dose group) and 150 mg O/50 mg A TID (for the higher dose group) were reached. The dosing regimen was as follows:

| Target dose | Week 1 and 2 | Week 3 and 4 | Week 5 and 6 | Week 7 to 26 |
|---|---|---|---|---|
| EMP16-02 | 60 mg O/20 mg A | 90 mg O/30 mg A | 120 mg O/40 mg A | 120 mg O/40 mg A |
| 120 mg O/40 mg A | | | | |
| EMP16-02 | 60 mg O/20 mg A | 90 mg O/30 mg A | 120 mg O/40 mg A | 150 mg O/50 mg A |
| 150 mg O/50 mg A | | | | |

Placebo treatment consisted of matching oral capsules. Placebo and EMP16-02 capsules needed to be taken TID together with three daily meals.

Patients came to the clinic at Visit 3 (week 7), Visit 4 (week 14) and Visit 5 (week 26) for safety assessments and assessments of weight and anthropometric measurements. Patients arrived in the morning after at least 8 hours overnight fasting. All visits started with a brief physical examination followed by blood sampling (fasting) and assessment of body weight and body composition. A standardised breakfast was served during which the patient took the IMP. All or a selection of the questionnaires, including the satiety and craving questionnaire, were filled in in a similar way as during Visit 2.

After 18 and 22 weeks of treatment (Day 123 +3 days and Day 151 +3 days, respectively), patients were asked to answer questions about IMP compliance, occurrence of AEs and use of concomitant medication using an electronic diary.

New IMP were handed out to the patients at Visit 2, 3 and 4.

At Visit 5 (week 26), the patients would take the last dose during breakfast at the clinic. Visit 6 was a safety follow up visit. Visit 7 was a 6 months follow-up visit for consenting patients who had completed the 26-week treatment period with active EMP16-02 treatment (120 mg O/40 mg A and 150 mg O/50 mg A) or placebo.

### Duration of Treatment

Each patient received 3 daily doses of EMP16 or matching placebo for 26 weeks. The IMP was taken together with the 3 main daily meals

### Efficacy Assessments

Weight, other anthropometric measurements (BMI, waist circumference, sagittal diameter, bio-impedance), blood sampling for fasting lipid metabolism, glucose metabolism and inflammations markers, blood pressure, questionnaires (meal pattern, GSRS, satiety and craving, activity and sleep and health and quality of life [RAND-36]), drop-out rate (assessed both in terms of safety and efficacy).

Meal pattern, satiety and craving and health and quality of life (RAND-36) questionnaires were analyzed using the Wilcoxon Rank Sum test. The GSRS questionnaire was analyzed using ANCOVA while the activity and sleep questionnaire was analyzed using a Chi-square test. The drop-out rate (overall and GI-related) following treatment with EMP16-120/40 or EMP16-150/50 as compared to placebo was analyzed using Chi-square test without continuity correction.

### Summary of Results

### Efficacy Results

Patients treated with EMP16 for 26 weeks lost significantly more weight than did patients who received placebo (p<0.0001 for both active treatment groups versus placebo at week 26). Mean relative weight loss was −5.8% and −6.5% after 26 weeks of treatment with EMP16-120/40 or EMP16-150/50 as compared to −0.7% in the placebo group at week 26 and mean absolute weight loss was −5.75 kg, −6.44 kg and −0.78 kg in the EMP16-120/40, EMP16-150/50 and placebo groups, respectively, at week 26. The same trends were observed also after 14 weeks of treatment as well as for females treated with EMP16-120/40 or EMP16-150/50 and for males treated with EMP16-150/50 at 14 and 26 weeks.
More patients in the active treatment groups had lost ≥5% (p<0.0001 for both active treatment groups vs placebo) and _≥10% (p_=0.0029 for EMP16-120/40 and p=0.0034 for EMP16-150/50 versus placebo) at week 26. In total, 55% and 67% of the patients in the EMP16-120/40 and EMP16-150/50 groups, respectively, lost ≥5% in weight between baseline and week 26 as compared to 13% in the placebo group. 23% and 22% in the active treatment groups lost ≥10% in weight as compared to 2.2% in the placebo group. The same trends were observed also after 14 weeks of treatment as well as for females treated with EMP16-120/40 or EMP16-150/50 at week 14 and week 26 but not for males treated with either dose.
The BMI and waist circumference were significantly more reduced in patients treated with EMP16 for 26 weeks than in patients who received placebo (BMI: p<0.0001, waist circumference: p=0.0087 for the EMP16-120/40 and p=0.0047 for EMP16-150/50 versus placebo). Mean relative and absolute change from baseline in BMI was −5.8% and −2.08 kg/m2 in the EMP16-120/40 group, −6.5% and −2.23 kg/m2 in the EMP16-150/50 group and −0.7% and −0.25 kg/m2 in the placebo group. Mean absolute change from baseline in waist circumference was −6.61 cm, −6.80 cm and −3.36 cm in the EMP16-120/40,
EMP16-150/50 and placebo groups, respectively at week 26. The same trends were observed also after 14 weeks of treatment for BMI. For waist circumference there were no statistically significant differences between the active treatment groups and placebo at week 14.
The sagittal diameter and body composition in terms of percentage body fat were significantly more reduced in patients treated with EMP16-150/50 for 26 weeks than in patients treated with placebo (sagittal diameter: p=0.0020, percentage body fat: p=0.0047 and p=0.0035 for relative and absolute change from baseline versus placebo, respectively). Mean absolute change from baseline in sagittal diameter was −1.89 cm in the EMP16-150/50 group and −0.49 cm in the placebo group at week 26. Mean relative and absolute change from baseline in percentage body fat was −5.4% and −2.21 in the EMP16-150/50 group and −0.3% and −0.19 in the placebo group, respectively. There were no statistically significant differences in sagittal diameter and body composition between patients treated with EMP16-120/40 and patients who received placebo at week 26. The same trends were observed also after 14 weeks of treatment except that there was a statistically significant difference in sagittal diameter between the EMP16-120/40 group and the placebo group at this time point.
In general, there were no significant differences between the active treatment groups and the placebo group in terms of satiety and craving and meal pattern. The appetite of the patients was not particularly affected over the course of the study in any treatment group and most patients appeared to, more or less, follow the recommendations for healthy eating habits already at baseline. Overall, there were minor and similar improvements in eating habits in terms of vegetables and root vegetables, fruits and berries and fish and seafood in all treatment groups. The eating habits in relation to sweet food (cookies, chocolate, sweets, chips, soft drinks) and breakfast habits were significantly improved in the EMP16-150/50 group compared to the placebo group at week 26.
There were no clinically relevant changes from baseline in fasting glucose metabolism markers (glucose, insulin, HbA1c), albumin or CRP in any treatment group over time. The lipid metabolism markers LDL cholesterol, HDL cholesterol and cholesterol, but not triglycerides, decreased more over time in the active treatment groups than in the placebo group. While the differences compared to placebo were statistically significant at most time points analysed, the small changes from baseline were not considered as clinically relevant. Also, ApoA1 and ApoB, appeared to decrease more compared to baseline in the active treatment groups than in the placebo group but the changes from baseline were not considered to be clinically relevant. Similarly, there were no clinically relevant changes from baseline in liver enzymes (ALT, AST, ALP or GGT) in any treatment group.
There were no statistically significant differences between the active treatment groups and the placebo group in terms of diabetic and pre-diabetic status at any timepoint. The proportions of diabetic patients at baseline and week 26 were 9.6% and 4.5% in the EMP16-120/40 group, 3.8% and 2.2% in the EMP16-150/50 group and 9.6% and 13% in the placebo group while the proportions of prediabetic patients at the corresponding time points were 25% and 20% in the EMP16-120/40 group, 27% and 22% in the EMP16-150/50 group and 37% and 22% in the placebo group.
There were no clinically relevant changes from baseline in blood pressure in any treatment group over time. Mean absolute changes from baseline in systolic blood pressure ranged between −2.7 and −2.2 mmHg in the EMP-120/40 group, −6.9 and −3.2 mmHg in the EMP-150/50 group and −2.2 and −1.8 mmHg in the placebo group. Mean absolute changes from baseline in diastolic blood pressure ranged between −3.0 and −2.4 mmHg in the EMP-120/40 group, −3.3 and −1.0 in the EMP-150/50 group and −1.5 and 0.7 mmHg in the placebo group.
Health-related quality of life based on the RAND-36 health survey improved more in the active treatment groups compared to the placebo group between baseline and week 26. A significant increase in the categories physical functioning and general health occurred in both active treatment groups compared to placebo group. In addition, patients in the EMP 150/50 group improved significantly more than patients in the placebo group in terms of bodily pain, energy/fatigue and emotional well-being. The mean overall health transition score increased by 18.5 and 16.5 points in the EMP-120/40 and EMP-150/50 groups, respectively, which was significantly more than the 5.9 points increase in the placebo group (p=0.0058 for EMP16-120/40 and p=0.0111 for EMP150/50 versus placebo). There were no significant differences between the active treatment groups and placebo in terms of changes in sleep patterns or performance of heavy physical work.
The overall drop-out frequency was comparable between treatment groups; 15.4%, 13.5% and 11.5% of the patients in the EMP16-120/40, EMP16-150/50 and placebo groups, respectively, discontinued early.

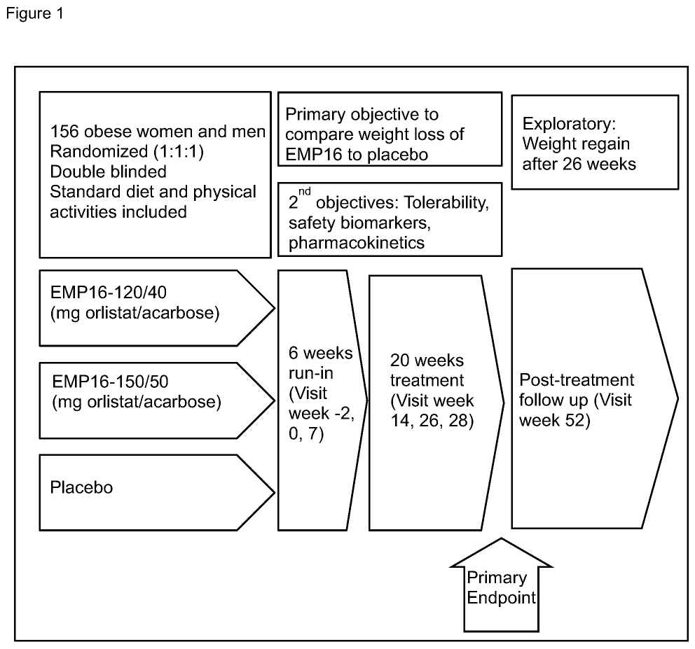

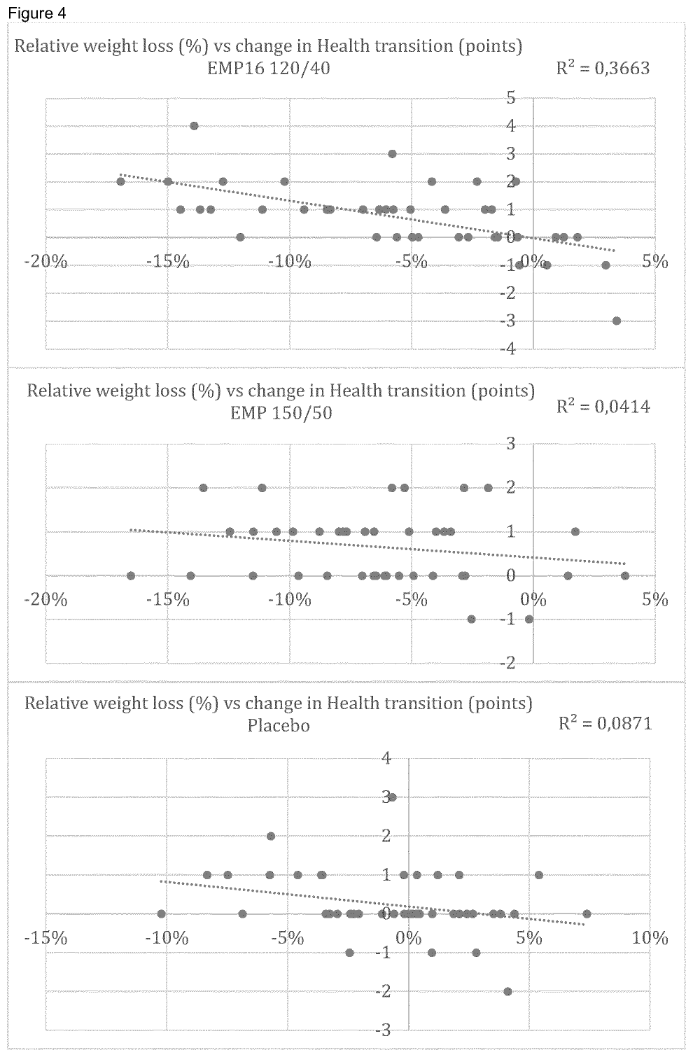

The results are shown in FIGS. 1-4 and in the following tables:

TABLE 1
Baseline characteristics of all randomised participants

| | EMP16-120/40 | | EMP16-150/50 | | Placebo | |
|---|---|---|---|---|---|---|
| | (n = 52) | | (n = 52) | | (n = 52) | |
| Age (years) | 49.4 | (12.2) | 50.7 | (13.6) | 49.5 | (12.8) |
| Female | 38 | (73%) | 36 | (69%) | 37 | (71%) |
| Male | 14 | (27%) | 16 | (31%) | 15 | (29%) |
| Body weight (kg) | 99.0 | (12.1) | 101.0 | (13.4) | 103.6 | (16.1) |
| BMI (kg/m²) | 35.1 | (3.3) | 34.6 | (3.6) | 36.2 | (4.5) |
| Body fat (%) | 41.3 | (7.5) | 41.1 | (7.4) | 41.9 | (6.9) |
| Waist circumference (cm) | 110.9 | (9.2) | 112.3 | (8.8) | 113.8 | (12.1) |
| Sagittal abdominal diameter (cm) | 27.2 | (2.3) | 27.3 | (2.5) | 28.0 | (3.4) |
| Systolic BP (mmHg) | 132.8 | (13.4) | 136.0 | (13.8) | 133.4 | (13.4) |
| Diastolic BP (mmHg) | 86.7 | (8.7) | 86.2 | (8.4) | 85.5 | (7.8) |
| Pulse | 67.8 | (8.9) | 66.8 | (10.5) | 69.5 | (10.7) |
| Fasting glucose (mmol/L) | 5.93 | (0.76) | 5.94 | (0.58) | 6.06 | (0.79) |
| HbA1c (mmol/mol) | 36.6 | (4.0) | 36.8 | (3.8) | 37.2 | (4.8) |
| Fasting insulin (mIU/L) | 18.54 | (12.08) | 17.71 | (10.17) | 21.53 | (17.38) |
| Fasting triglycerides (mmol/L) | 1.63 | (0.74) | 1.59 | (0.81) | 1.83 | (1.16) |
| Total cholesterol (mmol/L) | 5.56 | (1.15) | 5.26 | (1.20) | 5.35 | (0.86) |
| LDL cholesterol (mmol/L) | 3.65 | (0.96) | 3.37 | (1.07) | 3.44 | (0.85) |
| HDL cholesterol (mmol/L) | 1.31 | (0.27) | 1.32 | (0.30) | 1.29 | (0.34) |

Data are mean (SD) or n (%).
One participant was Asian and one was African American in the EMP16 120/40 group, and the remainder were white.
BMI = body-mass index.
BP = blood pressure.
HbA1c = glycosylated haemoglobin A1c.
HDL = high-density lipoprotein.
LDL = low-density lipoprotein.

TABLE 2
Estimated changes in body weight from
baseline to week 14 and week 26

| | | Estimated | | Estimated | |
|---|---|---|---|---|---|
| | | difference | | difference | |
| | | (95% CI) | | (95% CI) | |
| | EMP16- | EMP16- | EMP16- | EMP16- | |
| | 120/40 | 120/40 vs | 150/50 | 150/50 vs | Placebo |
| | (n = 48) | placebo | (n = 50) | placebo | (n = 51) |
| Absolute | −4.14 | −3.07 (−3.96 | −4.77 | −3.70 (−4.55 | −1.07 |
| weight change | (3.15) | to −2.18) | (3.07) | to −2.85) | (3.28) |
| (kg) from | | | | | |
| baseline to | | | | | |
| week 14 | | | | | |
| Relative weight | −4.20 | −3.23 (−4.11 | −4.69 | −3.73 (−4.53 | −0.97 |
| change (%) | (3.09)† | to −2.36) | (2.89)† | to −2.93) | (3.10) |
| from baseline | | | | | |
| to week 14 | | | | | |
| Loss of ≥5% | 31%¥ | 2.86 (1.05 | 42%‡ | 4.55 (1.72 | 14% |
| bodyweight at | | to 7.80) | | to 12.07) | |
| week 14¹ | | | | | |
| Loss of ≥10% | 8% | — | 4% | — | 0% |
| bodyweight at | | | | | |
| week 14 | | | | | |
| Absolute | −6.03 | −4.56 (−6.04 | −6.40 | −5.36 (−6.59 | −0.82 |
| weight change | (5.35) | to −3.09) | (4.80) | to −4.12) | (3.88) |
| (kg) from | | | | | |
| baseline to | | | | | |
| week 26 | | | | | |
| Relative weight | −5.53 | −4.70 (−6.16 | −6.25 | −5.42 (−6.60 | −0.83 |
| change (%) | (5.15)† | to −3.24) | (4.26)† | to −4.24) | (3.64) |
| from baseline | | | | | |
| to week 26 | | | | | |
| Loss of ≥5% | 50%† | 6.29 (2.36 | 64%† | 11.17 (4.17 | 14% |
| bodyweight at | | to 16.71) | | to 29.91) | |
| week 26 | | | | | |
| Loss of ≥10% | 21%‡ | 13.16 (1.62 | 20%‡ | 12.50 (1.53 | 2% |
| bodyweight at | | to 107.16) | | to 101.80) | |
| week 26 | | | | | |

Mean (SD) data and estimated difference (95% confidence interval) from the ITT analysis set with LOCF imputation.
ITT = intention-to-treat population, everyone with at least one post first dose measurement.
LOCF = last-observation-carried-forward.
SD = standard deviation Analysed with ANCOVA with LCOF imputation of missing data.
¹Percentages of the populations losing ≥5% or ≥10% bodyweight were analysed using Chi² and are presented as the proportions of participants (%) and odds ratios.
‡p < 0.01 for being different from placebo
†p < 0.0001 for being different from placebo

TABLE 3
Secondary outcome variables, absolute changes from baseline to week 26

| | | Estimated | | | Estimated | | |
|---|---|---|---|---|---|---|---|
| | | difference | | | difference | | |
| | | (95% CI) | P-value, | | (95% CI) | P-value, | |
| | | EMP16- | EMP16- | | EMP16- | EMP16- | |
| | EMP16- | 120/40 | 120/40 | EMP16- | 150/50 | 150/50 | |
| | 120/40 | vs | vs | 150/50 | vs | vs | Placebo |
| | (n = 44) | placebo | placebo | (n = 44) | placebo | placebo | (n = 46) |
| Body Mass | −2.07 | −1.80 | <0.001 | −2.22 | −1.95 | <0.001 | −0.27 |
| Index (kg/m²) | (1.97) | (−2.50 to | | (1.54) | (−2.55 to | | (1.33) |
| | | −1.10) | | | −1.35) | | |
| Body fat (%) | −1.16 | −1.11 | 0.0676 | −1.94 | −1.89 | 0.004 | −0.05 |
| | (3.83) | (−2.56 to | | (2.69) | (−3.09 to | | (3.05) |
| | | 0.34) | | | −0.69) | | |
| Waist | −6.30 | −3.15 | 0.009 | −7.17 | −4.02 | 0.005 | −3.15 |
| circumference | (5.70) | (−5.44 to | | (5.21) | (−6.21 to | | (5.26) |
| (cm) | | −0.86) | | | −1.83) | | |
| Sagittal | −1.3 | −0.90 | 0.052 | −1.8 | −1.40 | 0.002 | −0.4 |
| abdominal | (2.2) | (−1.80 to | | (2.0) | (−2.26 to | | (2.1) |
| diameter | | 0.00) | | | −0.54) | | |
| (cm) | | | | | | | |
| Systolic | −2.7 | −0.90 | 0.577 | −4.9 | −3.10 | 0.182 | −1.8 |
| blood | (12.0) | (−5.75 to | | (13.6) | (−8.30 to | | (11.2) |
| pressure | | 3.95) | | | 2.10) | | |
| (mmHg) | | | | | | | |
| Diastolic | −3.0 | −1.50 | 0.377 | −3.0 | −1.50 | 0.384 | −1.5 |
| blood | (7.0) | (−4.49 to | | (8.1) | (−4.72 to | | (7.3) |
| pressure | | 1.49) | | | 1.72) | | |
| (mmHg) | | | | | | | |
| Pulse | 0.6 | −0.70 | 0.684 | −1.2 | −2.50 | 0.184 | 1.3 |
| (beats/min) | (6.9) | (−3.96 to | | (9.1) | (−6.20 to | | (8.6) |
| | | 2.56) | | | 1.20) | | |
| Fasting | −0.27 | −0.11 | 0.342 | −0.26 | −0.10 | 0.365 | −0.16 |
| glucose | (0.50) | (−0.32 to | | (0.65) | (−0.34 to | | (0.49) |
| (mmol/L) | | 0.10) | | | 0.14) | | |
| HbA1c | −1.2 | −0.60 | 0.291 | −1.1 | −0.50 | 0.342 | −0.6 |
| (mmol/mol) | (2.3) | (−1.54 to | | (2.4) | (−1.46 to | | (2.2) |
| | | 0.34) | | | 0.46) | | |
| Fasting | −3.62 | −2.11 | 0.397 | 0.08 | 1.59 | 0.591 | −1.51 |
| insulin | (9.71) | (−5.90 to | | (18.10) | (−4.30 to | | (8.41) |
| (mIU/L) | | 1.68) | | | 7.48) | | |
| Fasting | −0.09 | 0.15 | 0.439 | −0.03 | 0.21 | 0.191 | −0.24 |
| triglycerides | (0.64) | (−0.15 to | | (0.63) | (−0.09 to | | (0.78) |
| (mmol/L) | | 0.45) | | | 0.51) | | |
| Total | −0.53 | −0.40 | 0.007 | −0.45 | −0.32 | 0.030 | −0.13 |
| cholesterol | (0.73) | (−0.68 to | | (0.76) | (−0.60 to | | (0.59) |
| (mmol/L) | | −0.12) | | | −0.04) | | |
| LDL | −0.34 | −0.35 | 0.002 | −0.26 | −0.27 | 0.020 | 0.01 |
| (mmol/L) | (0.52) | (−0.57 to | | (0.61) | (−0.51 to | | (0.55) |
| | | −0.13) | | | −0.03) | | |
| HDL | −0.11 | −0.09 | 0.025 | −0.16 | −0.14 | <0.001 | −0.02 |
| (mmol/L) | (0.16) | (−0.16 to | | (0.19) | (−0.22 to | | (0.18) |
| | | −0.02) | | | −0.06) | | |

Observed mean data (SD) and estimated difference (95% confidence intervals) are presented for the ITT analysis set without imputations. Analysis of covariance was performed with imputations using last observation carried forward (LOCF). Changes at weeks 7 and 14 during the trial are presented in the appendix.
HbA1c = glycosylated haemoglobin A1C. HDL = high density lipoprotein. ITT = intention-to-treat population. LDL = low density lipoprotein. LOCF = last-observation-carried-forward. SD = standard deviation.

TABLE 4
Questionnaires, absolute changes from baseline to week 26

| | | Estimated | | | Estimated | | |
|---|---|---|---|---|---|---|---|
| | | difference | | | difference | | |
| | | (95% CI) | P-value, | | (95% CI) | P-value, | |
| | | EMP16- | EMP16- | | EMP16- | EMP16- | |
| | EMP16- | 120/40 | 120/40 | EMP16- | 150/50 | 150/50 | |
| | 120/40 | vs | vs | 150/50 | vs | vs | Placebo |
| | (n = 44) | placebo | placebo | (n = 44) | placebo | placebo | (n = 46) |
| Satiety and craving | 8.1 | 9.00 | | −0.8 | 0.10 | | −0.9 |
| total score | (35.4) | (−5.74 to | | (29.7) | (−13.50 to | | (35.2) |
| | | 23.74) | | | 13.70) | | |

RAND-36

| | | | | | | | |
|---|---|---|---|---|---|---|---|
| 1. Physical | 9.0 | 7.00 | 0.008 | 11.5 | 9.50 | 0.002 | 2.0 |
| functioning | (14.9) | (1.48 to | | (15.3) | (3.88 to | | (11.3) |
| | | 12.52) | | | 15.12) | | |
| 2. Role | 7.7 | 7.70 | 0.181 | 12.2 | 12.20 | 0.075 | 0.0 |
| functioning/ | (36.0) | (−5.95 to | | (35.8) | (−1.40 to | | (29.0) |
| Physical | | 21.35) | | | 25.80) | | |
| 3. Pain (Bodily | 1.3 | 1.50 | 0.877 | 11.8 | 12.00 | 0.013 | −0.2 |
| Pain) | (17.4) | (−5.68 to | | (21.0) | (4.02 to | | (17.0) |
| | | 8.68) | | | 19.98) | | |
| 4. General health | 3.3 | 8.00 | 0.017 | 6.0 | 10.70 | <0.001 | −4.7 ± |
| | (17.1) | (1.68 to | | (12.6) | (5.38 to | | 12.9) |
| | | 14.32) | | | 16.02) | | |
| 5. Energy/fatigue | −1.4 | 1.70 | 0.437 | 7.2 | 10.30 | 0.022 | −3.1 |
| (Vitality) | (19.9) | (−6.01 to | | (20.0) | (2.57 to | | (16.9) |
| | | 9.41 | | | 18.03) | | |
| 6. Social | 1.2 | 3.90 | 0.468 | 6.4 | 9.10 | 0.089 | −2.7 |
| Functioning | (20.3) | (−4.37 to | | (26.8) | (−0.65 to | | (19.3) |
| | | 12.17) | | | 18.85) | | |
| 7. Role | −2.4 | 8.90 | 0.286 | 3.3 | 14.60 | 0.145 | −11.3 |
| functioning/ | (38.5) | (−5.75 to | | (43.3 | (−1.17 to | | (31.3) |
| Emotional | | 23.55) | | | 30.37) | | |
| 8. Emotional well- | −3.8 | 0.50 | 0.302 | 1.7 | 6.00 | 0.036 | −4.3 |
| being (Mental) | (16.6) | (−5.41 to | | (17.8) | (−0.21 to | | (11.2) |
| | | 6.41) | | | 12.21) | | |
| 9. Health | 18.5 | 12.60 | 0.006 | 16.5 | 10.60 | 0.011 | 5.9 |
| Transition score | (29.8) | (2.05 to | | (20.6) | (2.18 to | | (19.7) |
| | | 23.15) | | | 19.02) | | |

GSRS

| | | | | | | | |
|---|---|---|---|---|---|---|---|
| Diarrhoea | 1.3 | 1.00 | <0.001 | 1.8 | 1.50 | <0.001 | 0.3 |
| syndrome | (1.5) | (0.50 to | | (1.4) | (1.02 to | | (0.8) |
| | | 1.50) | | | 1.98) | | |
| Indigestion | 0.8 | 0.70 | <0.001 | 1.0 | 0.90 | <0.001 | 0.1 |
| syndrome | (1.0) | (0.34 to | | (1.1) | (0.52 to | | (0.7) |
| | | 1.06) | | | 1.28) | | |
| Constipation | 0.2 | 0.10 | 0.831 | 0.3 | 0.20 | 0.404 | 0.1 |
| syndrome | (1.0) | (−0.23 to | | (0.6) | (−0.03 to | | (0.5) |
| | | 0.43) | | | 0.43) | | |
| Abdominal pain | 0.2 | 0.30 | 0.079 | 0.1 | 0.20 | 0.284 | −0.1 |
| syndrome | (0.7) | (0.03 to | | (0.8) | (−0.10 to | | (0.6) |
| | | 0.57) | | | 0.50) | | |
| Reflux syndrome | 0.0 | −0.20 | 0.717 | −0.1 | −0.30 | 0.110 | 0.2 |
| | (0.9) | (−0.50 to | | (0.4) | (−0.49 to | | (0.5) |
| | | 0.10) | | | −0.11) | | |

Observed mean data (SD) and estimated difference (95% confidence intervals) are presented for the ITT analysis set without imputations. Analysis of covariance was performed with imputations using last observation carried forward (LOCF). Changes at weeks 7 and 14 during the trial are presented in the appendix. GSRS = gastrointestinal symptoms rating scale, where higher scores indicate increased intensity. RAND-36 = 36-item short form health survey where higher score indicates better quality of life. ITT = intention-to-treat population. SD = standard deviation.

TABLE 5
Participant withdrawal and adverse events reported
by ≥5% of participants in any group

| | EMP16-120/40 | | EMP16-150/50 | | Placebo | |
|---|---|---|---|---|---|---|
| | (n = 52) | | (n = 52) | | (n = 52) | |
| Overall withdrawal rate | 8 | (15%) | 7 | (14%) | 6 | (12%) |
| Any AE | 32 | (62%) | 39 | (75%) | 30 | (58%) |

| | | | | | |
|---|---|---|---|---|---|
| Any AE leading to | 4 | (8%) | 6 | (12%) | 0 |
| withdrawal | | | | | |

Most frequent AEs by MedDRA PT*

| | | | |
|---|---|---|---|
| Nasopharyngitis | 4 (8%) 4 | 10 (19%) 10 | 13(25%) 15 |
| Diarrhoea | 8 (15%) 9 | 8 (15%) 9 | 0 |
| Headache | 4 (8%) 5 | 2 (4%) 2 | 3 (6%) 4 |
| Flatulence | 4 (8%) 4 | 3 (6%) 4 | 1 (2%) 1 |
| COVID-19 | 0 | 6 (12%) 6 | 2 (4%) 2 |
| Abdominal distension | 4 (8%) 4 | 1 (2%) 1 | 0 |

Causality

| | | | | | | |
|---|---|---|---|---|---|---|
| Unlikely | 22 | (42%) | 28 | (54%) | 27 | (52%) |
| Possibly | 7 | (13%) | 9 | (17%) | 6 | (12%) |
| Probably | 13 | (25%) | 12 | (23%) | 2 | (4%) |

Severity

| | | | | | | |
|---|---|---|---|---|---|---|
| Mild | 21 | (40%) | 24 | (46%) | 22 | (42%) |
| Moderate | 11 | (21%) | 13 | (25%) | 11 | (21%) |
| Severe | 14 | (27%) | 12 | (23%) | 3 | (6%) |

Data are shown as number of participants (percentage of treatment arm) and number of events.
Data are from the safety population, all participants who were randomized exposed to at least one treatment dose.
AE = adverse event.
MedDRA = Medical Dictionary for Regulatory Activities.
PT = preferred term.

### Appendix Tables

TABLE A2
Additional baseline characteristics of all
randomised participants (Safety population)

| | EMP16-120/40 | | EMP16-150/50 | | Placebo | |
|---|---|---|---|---|---|---|
| | (n = 52) | | (n = 52) | | (n = 52) | |
| ALT | 0.544 | (0.332) | 0.519 | (0.282) | 0.534 | (0.260) |
| AST | 0.446 | (0.138) | 0.445 | (0.134) | 0.483 | (0.156) |
| ALP | 1.187 | (0.317) | 1.220 | (0.380) | 1.191 | (0.313) |
| GGT | | | | | | |
| Albumin | 40.1 | (3.5) | 39.4 | (3.5) | 39.6 | (3.3) |
| Hs-CRP | 4.6 | (4.7) | 4.7 | (6.8) | 5.4 | (7.5) |

Glucose tolerance status¹

| | | | | | | |
|---|---|---|---|---|---|---|
| Diabetes Prediabetes | 5 | (9.6%) | 2 | (3.8%) | 5 | (9.6%) |
| Normoglycaemic | 13 | (25%) | 14 | (27%) | 19 | (37%) |
| | 34 | (65.4%) | 36 | (70.8%) | 28 | (53.4%) |
| Patients on BP | 14 | (27%) | 16 | (31%) | 14 | (27%) |
| medication | | | | | | |
| Patients on lipid | 1 | (2%) | 7 | (13%) | 3 | (6%) |
| lowering medication | | | | | | |
| (e.g. statins) | | | | | | |

ALT: Alanine Aminotransferase,
AST: Aspartate Aminotransferase,
ALP: Alkaline Phosphatase,
GGT: Gamma Glutamyl Transferase

TABLE A3
Questionnaires, baseline data [mean points (SD)]

| | EMP16 120 mg/40 mg | | EMP16 150 mg/50 mg | | PLACEBO | |
|---|---|---|---|---|---|---|
| Satiety and Craving total score¹ | 101.3 | (27.2) | 107.9 | (30.2) | 120.2 | (31.8) |
| Meal Pattern, total score² | 9.5 | (2.3) | 8.3 | (2.6) | 9.1 | (2.5) |
| RAND-36³ | | | | | | |
| 1. Physical functioning | 80.8 | (17.2) | 79.4 | (19.1) | 81.0 | (16.5) |
| 2. Role functioning/Physical | 80.3 | (33.7) | 78.4 | (31.3) | 87.0 | (24.0) |
| 3. Pain (Bodily Pain) | 82.6 | (19.7) | 71.8 | (20.0) | 80.6 | (19.9) |
| 4. General health | 69.8 | (16.9) | 71.5 | (17.6) | 73.1 | (15.6) |
| 5. Energy/fatigue (Vitality) | 63.7 | (17.8) | 57.5 | (18.7) | 64.8 | (17.5) |
| 6. Social Functioning | 87.3 | (14.7) | 82.9 | (22.3) | 87.5 | (18.4) |
| 7. Role functioning/Emotional | 80.8 | (33.2) | 77.6 | (34.8) | 91.0 | (23.9) |
| 8. Emotional well-being (Mental) | 78.7 | (16.2) | 77.3 | (14.9) | 81.1 | (12.2) |
| 9. Health Transition score | 49.0 | (17.8) | 48.6 | (16.7) | 50.5 | (14.4) |
| GSRS⁴ | | | | | | |
| Diarrhoea syndrome | 1.4 | (0.7) | 1.4 | (0.7) | 1.2 | (0.4) |
| Indigestion syndrome | 1.9 | (0.8) | 1.9 | (0.7) | 1.8 | (0.6) |
| Constipation syndrome | 1.5 | (0.8) | 1.3 | (0.5) | 1.2 | (0.4) |
| Abdominal pain syndrome | 1.5 | (0.6) | 1.6 | (0.6) | 1.5 | (0.5) |
| Reflux syndrome | 1.3 | (0.7) | 1.3 | (0.5) | 1.2 | (0.4) |

¹Sum of five different satiety and craving related questions
²Sum of seven questions related to meal pattern
³Quality of Life instrument
⁴Gastrointestinal symptoms rating scale

TABLE A4
The primary outcome variable weight loss, relative change
from baseline to weeks 14 and 26 in the Per Protocol
population and a post-hoc imputation population.

| | | | |
|---|---|---|---|
| Popu- | | | |
| lation | EMP16-120/40 | EMP16-150/50 | Placebo |

Relative weight change (%; mean (SD)
from baseline to week 14

| | | | | | |
|---|---|---|---|---|---|
| PP | −4.52 | (3.22)† | −4.72 | (3.05)† | −0.84 (3.25) |
| PHIP | −4.37 | (3.09)† | −4.78 | (2.98)† | −1.15 (2.87) |

Estimated difference to placebo (%;
estimated mean [95% Cl]) at week 14

| | | | | | |
|---|---|---|---|---|---|
| PP | −3.67 | (−4.66 to −2.69) | −3.87 | (−4.80 to −2.94) | — |
| PHIP | −3.21 | (−4.10 to −2.33) | −3.62 | (−4.45 to −2.80) | — |

Relative weight change (%; mean
(SD) from baseline to week 26

| | | | | | |
|---|---|---|---|---|---|
| PP | −6.15 | (5.26)† | −6.49 | (4.57)† | −0.78 (3.86) |
| PHIP | −5.76 | (5.19)† | −6.25 | (4.40)† | −1.28 (3.66) |

Estimated difference to placebo (%;
estimated mean [95% Cl]) at week 26

| | | | | | |
|---|---|---|---|---|---|
| PPP | −5.37 | (−3.76 to −6.98) | −5.71 | (−4.31 to −7.11) | — |
| PHIP | −4.47 | (−5.9 to −2.99) | −4.96 | (−6.18 to −3.74) | — |

Relative weight loss data are observed means (SD) using available data from the PP = per-protocol population and the PHIP = post-hoc imputation population.
Number of participants in PP were: 41 (EMP16-120/40), 41 (EMP16-150/50) and 40 (Placebo). Number of participants in the PHIP were: 48 (EMP16-120/40), 50 (EMP16-150/50) and 51 (Placebo).
†p < 0.0001 for being different from placebo

TABLE A5
Anthropometric secondary outcome variables and vital
signs, absolute change from baseline to week 14.

| | | | P-value, | | | P-value, | | |
|---|---|---|---|---|---|---|---|---|
| | | | EMP16- | | | EMP16- | | |
| | EMP16- | | 120/40 vs | EMP16- | | 150/50 vs | Placebo | |
| | 120/40 | | placebo | 150/50 | | placebo | (n = 46) | |
| BMI | −1.55 | (1·18) | <.001 | −1.67 | (1.08) | <.001 | −0.40 | (1.17) |
| Waist | −3.92 | (6.02) | 0.505 | −3.83 | (4.11) | 0.555 | −3.22 | (4.67) |
| circumference | | | | | | | | |
| Sagittal diameter | −1.79 | (2.02) | 0.008 | −1.71 | (2.16) | 0.014 | −0.75 | (1.85) |
| Body fat | −0.40 | (3.33) | 0.273 | −1.11 | (2.24) | 0.020 | 0.15 | (2.40) |
| percentage | | | | | | | | |
| Systolic blood | −2.6 | (9.9) | 0.733 | −6.9 | (13.4) | 0.030 | −2.2 | (8.9) |
| pressure (mmHg) | | | | | | | | |
| Diastolic blood | −2.8 | (7.0) | 0.054 | −3.3 | (8.7) | 0.022 | 0.2 | (5.9) |
| pressure (mmHg) | | | | | | | | |
| Pulse | −2.25 | (6.25) | 0.983 | −3.81 | (8.93) | 0.326 | −2.22 | (6.15) |

TABLE A6
Secondary outcome variables from blood samples, absolute
change from baseline to week 7 and week 14.

| | EMP16- | | EMP16- | | Placebo |
|---|---|---|---|---|---|
| | 120/40 | | 150/50 | | ( n =51) |
| n at week 7 | | | 50 | | 50 |
| n at week 14 | | | 46 | | 49 |
| n at week 26 | | | 45 | | 46 |
| HbA1c (mmol/mol), | −1.5 | 0.028 | −0.9 | 0.512 | −0.7 |
| week 7 | (2·3) | | (1.7) | | (1.4) |
| HbA1c (mmol/mol), | −0.9 | 0.067 | −0.3 | 0.584 | 0.0 |
| week 14 | (2·3) | | (1.9) | | (1.9) |
| Fasting glucose | −0.16 | 0.478 | −0.17 | 0.426 | −0.10 |
| (mmol/L) week 7 | (0.45) | | (0.63) | | (0.40) |
| Fasting glucose | −0.30 | 0.186 | −0.27 | 0.248 | −0.15 |
| (mmol/L) week 14 | (0.50) | | (0.55) | | (0.45) |
| Fasting insulin (mIU/L) | −3.958 | 0.638 | −3.129 | 0.978 | −3.320 |
| week 7 | (8.125) | | (8.642) | | (10.68) |
| Fasting insulin (mIU/L) | −4.22 | 0.036 | −3.24 | 0.101 | −0.01 |
| week 14 | (7.67) | | (7.92) | | (11.83) |
| Fasting triglycerides | 0.011 | 0.249 | −0.047 | 0.451 | −0.149 |
| (mmol/L) week 7 | (0.632) | | (0.619) | | (0.659) |
| Fasting triglycerides | −0·116 | 0.327 | −0.070 | 0.145 | −0.275 |
| (mmol/L) week 14 | (0.483) | | (0.623) | | (0−732) |
| Total cholesterol | −0.59 | <.0001 | −0.48 | 0.001 | 0.00 |
| (mmol/L) week 7 | (0.82) | | (0.59) | | (0.53) |
| Total cholesterol | −0.72 | 0.001 | −0.56 | 0.014 | −0.22 |
| (mmol/L) week 14 | (0.70) | | (0.76) | | (0.56) |
| LDL (mmol/L) week 7 | −0.49 | <.001 | −0.34 | 0.005 | −0.05 |
| | (0.52) | | (0.52) | | (0.51) |
| LDL (mmol/L) week 14 | −0.51 | 0.001 | −0.36 | 0.047 | −0.14 |
| | (0.46) | | (0.66) | | (0.51) |
| HDL (mmol/L) week 7 | −0.15 | <.0001 | −0.16 | <0.0001 | 0.03 |
| | (0.14) | | (0.16) | | (0.19) |
| HDL (mmol/L) week 14 | −0.14 | 0.002 | −0.20 | <0.0001 | −0.02 |
| | (0.15) | | (0.19) | | (0.19) |

Glucose tolerance status at week 26, n (%) ¹

| | | | | | |
|---|---|---|---|---|---|
| Diabetes | 2 | 0.3409 | 1 | 0.1488 | 6 |
| | (4.5%) | | (2.2%) | | (13%) |
| Prediabetes | 9 | | 10 | | 10 |
| | (20%) | | (22%) | | (22%) |
| Normoglycemic | 33 | | 34 | | 30 |
| | (75%) | | (76%) | | (65%) |

Observed mean data (SD are presented for the ITT analysis set using LOCF imputation.
Treatment differences are presented as estimated means and 95% CI.
¹ Glucose tolerance status was defined as diabetes when fasting blood glucose ≥7.0 mmol/L and as prediabetes when fasting glucose was between 6.1 mmol/L and 7.0 mmol/L and was presented as n (%).

TABLE A7
Secondary safety outcome variables, baseline values
and absolute changes at week 7, 14 and 26.

| EMP16- | EMP16- | |
|---|---|---|
| 120/40 | 150/50 | Placebo |

| | | | |
|---|---|---|---|
| n at week 7 | 48 | 50 | 50 |
| n at week 14 | 45 | 46 | 49 |
| n at week 26 | 44 | 45 | 46 |

ALT (uKat/L)

| | | | | | | | | |
|---|---|---|---|---|---|---|---|---|
| Week 7 | 0.024 | (0.242) | 0.074 | 0.020 | (0.234) | 0.088 | −0.056 | (0.188) |
| Week 14 | −0.026 | (0.204) | 0.4925 | 0.043 | (0.366) | 0.0555 | −0.063 | (0.202) |
| Week 26 | −0.057 | (0.217) | 0.8744 | −0.015 | (0.313) | 0.3057 | −0.069 | (0.151) |

AST

| | | | | | | | | |
|---|---|---|---|---|---|---|---|---|
| Week 7 | 0.010 | (0.127) | 0.092 | 0.011 | (0.128) | 0.067 | −0.040 | (0.150) |
| Week 14 | −0.034 | (0.105) | 0.608 | 0.076 | (0.721) | 0.086 | −0.072 | (0.143) |
| Week 26 | −0.046 | (0.112) | 0.677 | −0.012 | (0.138) | 0.385 | −0.037 | (0.137) |

ALP

| | | | | | | | | |
|---|---|---|---|---|---|---|---|---|
| Week 7 | 0−031 | (0.136) | 0.068 | −0.003 | (0.128) | 0.598 | −0.016 | (0.121) |
| Week 14 | 0.035 | (0.135) | 0.082 | −0.001 | (0.163) | 0.534 | −0.021 | (0.141) |
| Week 26 | 0.090 | (0.178) | 0.037 | 0.036 | (0.178) | 0.559 | 0.013 | (0.134) |

GGT

| | | | | | | | | |
|---|---|---|---|---|---|---|---|---|
| Week 7 | −0.030 | (0.152) | 0.2200 | −0.047 | (0.192) | 0.3775 | −0.094 | (0.470) |
| Week 14 | −0.069 | (0.195) | 0.6703 | −0.018 | (0.223) | 0.2478 | −0.094 | (0.474) |
| Week 26 | −0.073 | (0.201) | 0.401 | −0.071 | (0.161) | 0.410 | −0.123 | (0.515) |

Albumin

| | | | | | | | | |
|---|---|---|---|---|---|---|---|---|
| Week 7 | 0.5 | (3.0) | 0.161 | 0.6 | (2.8) | 0.110 | −0.2 | (2.5) |
| Week 14 | −0.6 | (1.9) | 0.078 | −1.0 | (2.9) | 0.385 | −1.4 | (2·1) |
| Week 26 | −0.2 | (2.4) | 0.918 | −0.2 | (2.9) | 0.994 | −0.3 | (2.4) |

Hs-CRP

| | | | | | | | | |
|---|---|---|---|---|---|---|---|---|
| Week 7 | −0.7 | (3.2) | 0.733 | 0.1 | (2.6) | 0.695 | −0.2 | (7.5) |
| Week 14 | −0.7 | (3.1) | 0.8853 | −0.7 | (2.8) | 0.7778 | −0.3 | (7.3) |
| Week 26 | 0.7 | (11.0) | 0.346 | −1.1 | (2.0) | 0.825 | −0.6 | (6.6) |

TABLE A8
Satiety and craving - total score (mean ± SD)

| | EMP16 | | | EMP16 | | | | |
|---|---|---|---|---|---|---|---|---|
| VISITS | 120 mg/40 mg | | | 150 mg/50 mg | | | PLACEBO | |
| Baseline - Visit 2 | 101.3 | (27.2) | | 107.9 | (30.2) | | 120.2 | (31.8) |
| (points) | | | | | | | | |
| Week 7 - Visit 3¹ | 0.9 | (26.7) | 0.0606 | −3.5 | (31.0) | 0.4598 | −10.5 | (36.3) |
| Week 14 - Visit 4¹ | 2.1 | (33.7) | 0.3651 | −3.3 | (29.5) | 0.7100 | −5.6 | (41.6) |
| Week 26 - Visit 5¹ | 8.1 | (35.4) | 0.2287 | −0.8 | (29.7) | 0.9191 | −0.9 | (35.2) |

¹Absolute change from baseline

TABLE A9
Meal pattern - total score (mean ± SD).
Higher score indicates healthier eating pattern.

| | EMP16 | | EMP16 | | |
|---|---|---|---|---|---|
| VISITS | 120 mg/40 mg | | 150 mg/50 mg | | PLACEBO |
| Baseline - Visit 2 | 9.5 (2·3) | | 8.3 (2.6) | | 9.1 (2.5) |
| (points) | | | | | |
| Week 7 - Visit 31 | 1.1 (1.6) | 0.6575 | 1.8 (2.3) | 0.3170 | 1.3 (2.0) |
| Week 14 - Visit 4¹ | 1.3 (1.9) | 0.6813 | 1.6 (2·3) | 0.3568 | 1.0 (1.8) |
| Week 26 - Visit 5¹ | 1 .1 (2·3) | 0.9593 | 1.7 (2.4) | 0.2096 | 0.9 (1.8) |

¹Absolute change from baseline

TABLE A10
Meal pattern - How often do you eat cookies, chocolates, sweets, chips or soft
drinks (mean points ± SD). Higher score indicates decreased consumption.

| | EMP16 | | EMP16 | | |
|---|---|---|---|---|---|
| VISITS | 120 mg/40 mg | | 150 mg/50 mg | | PLACEBO |
| Baseline - Visit 2 | 2.1 ± 0.8 | | 1.5 ± 0.9 | | 1.8 ± 0.8 |
| Week 7 - Visit 3¹ | 0.3 ± 0.7 | 0.0529 | 0.8 ± 1.0 | 0.4962 | 0.6 ± 0.8 |
| Week 14 - Visit 4¹ | 0.3 ± 0.8 | 0.2738 | 0.6 ± 1.0 | 0.4371 | 0.5 ± 0.8 |
| Week 26 - Visit 5¹ | 0.3 ± 0.7 | 0.9739 | 0.8 ± 0.9 | 0.0086 | 0.3 ± 0.8 |

¹Absolute change from baseline

TABLE A11
Meal pattern - How often do you breakfast? (mean points ± SD).
Higher score indicates more frequent consumption of breakfast.

| | EMP16 | | EMP16 | | |
|---|---|---|---|---|---|
| VISITS | 120 mg/40 mg | | 150 mg/50 mg | | PLACEBO |
| Baseline - Visit 2 | 2.6 ± 0.7 | | 2.4 ± 0.8 | | 2.7 ± 0.6 |
| Week 7 - Visit 3¹ | 0.4 ± 0.8 | 0.2375 | 0.4 ± 0.7 | 0.1105 | 0.1 ± 0.5 |
| Week 14 - Visit 4¹ | 0.4 ± 0.8 | 0.0222 | 0.4 ± 0.7 | 0.0156 | 0.1 ± 0.5 |
| Week 26 - Visit 5¹ | 0.3 ± 0.8 | 0.1012 | 0.4 ± 0.7 | 0.0231 | 0.0 ± 0.4 |

¹Absolute change from baseline

TABLE A12
Sleep and activity questionnaire

| | | |
|---|---|---|
| EMP16 | EMP16 | |
| 120 mg/40 mg | 150 mg/50 mg | PLACEBO |

Did you have a normal night's sleep?

| | | | |
|---|---|---|---|
| Baseline | 41(79%)/52 | 40(77%)/52 | 41(79%)/52 |
| Week 7 | 39(89%)/44 | 37(93%)/40 | 38(86%)/44 |
| Week 14 | 31(72%)/43 | 40(89%)/45 | 40(83%)/48 |
| Week 26 | 34(81%)/42 | 36(86%)/42 | 40(85%)/47 |

Have you done any heavy physical work?

| | | | |
|---|---|---|---|
| Baseline | 15(29%)/52 | 10(19%)/52 | 9(17%)/52 |
| Week 7 | 8(18%)/44 | 6(15%)/40 | 9(20%)/44 |
| Week 14 | 10(23%)/43 | 8(18%)/45 | 7(15%)/48 |
| Week 26 | 10(24%)/42 | 6(14%)/42 | 6(13%)/47 |

Percentage of participants answering yes to stated questions. No significant differences between active arms and placebo (data not shown).

TABLE A13
Adverse events by system organ class and preferred term (Safety population)

| | EMP16-02 | | EMP16-02 | | | | | |
|---|---|---|---|---|---|---|---|---|
| | 120/40 | | 150/50 | | PLACEBO | | Total | |
| System organ class | N = 52 | | N = 52 | | N = 52 | | N = 156 | |
| Preferred term | n(%) | m | n(%) | m | n(%) | m | n(%) | m |
| Infections and infestations | 6(12%) | 8 | 17(33%) | 18 | 20(38%) | 22 | 43(28%) | 48 |
| COVID-19 | 0 | 0 | 6(12%) | 6 | 2(4%) | 2 | 8(5%) | 8 |
| Ear infection | 1(2%) | 1 | 0 | 0 | 0 | 0 | 1(1%) | 1 |
| Fungal skin infection | 1(2%) | 1 | 0 | 0 | 0 | 0 | 1(1%) | 1 |
| Gastroenteritis | 1(2%) | 1 | 0 | 0 | 0 | 0 | 1(1%) | 1 |
| Gastrointestinal infection | 0 | 0 | 0 | 0 | 1(2%) | 1 | 1(1%) | 1 |
| Herpes zoster | 0 | 0 | 0 | 0 | 1(2%) | 1 | 1(1%) | 1 |
| Influenza | 0 | 0 | 0 | 0 | 1(2%) | 1 | 1(1%) | 1 |
| Nasopharyngitis | 4(8%) | 4 | 10(19%) | 10 | 13(25%) | 15 | 27(17%) | 29 |
| Otitis media | 0 | 0 | 1(2%) | 1 | 0 | 0 | 1(1%) | 1 |
| Rhinitis | 1(2%) | 1 | 0 | 0 | 0 | 0 | 1(1%) | 1 |
| Upper respiratory tract infection | 0 | 0 | 0 | 0 | 2(4%) | 2 | 2(1%) | 2 |
| Urinary tract infection | 0 | 0 | 1(2%) | 1 | 0 | 0 | 1(1%) | 1 |
| Gastrointestinal disorders | 14(27%) | 27 | 16(31%) | 21 | 5(10%) | 8 | 35(22%) | 56 |
| Abdominal discomfort | 1(2%) | 1 | 1(2%) | 1 | 0 | 0 | 2(1%) | 2 |
| Abdominal distension | 4(8%) | 4 | 1(2%) | 1 | 0 | 0 | 5(3%) | 5 |
| Abdominal pain | 2(4%) | 2 | 0 | 0 | 0 | 0 | 2(1%) | 2 |
| Abdominal pain upper | 1(2%) | 1 | 1(2%) | 1 | 0 | 0 | 2(1%) | 2 |
| Anal incontinence | 0 | 0 | 1(2%) | 1 | 0 | 0 | 1(1%) | 1 |
| Constipation | 0 | 0 | 0 | 0 | 1(2%) | 1 | 1(1%) | 1 |
| Defaecation urgency | 1(2%) | 1 | 2(4%) | 2 | 1(2%) | 1 | 4(3%) | 4 |
| Diarrhoea | 8(15%) | 9 | 8(15%) | 9 | 0 | 0 | 16(10%) | 18 |
| Dyspepsia | 1(2%) | 1 | 0 | 0 | 1(2%) | 1 | 2(1%) | 2 |
| Encopresis | 1(2%) | 2 | 0 | 0 | 0 | 0 | 1(1%) | 2 |
| Flatulence | 4(8%) | 4 | 3(6%) | 4 | 1(2%) | 1 | 8(5%) | 9 |
| Food poisoning | 0 | 0 | 0 | 0 | 1(2%) | 1 | 1(1%) | 1 |
| Gastrointestinal motility disorder | 1(2%) | 1 | 0 | 0 | 0 | 0 | 1(1%) | 1 |
| Gastrooesophageal reflux disease | 1(2%) | 1 | 0 | 0 | 1(2%) | 1 | 2(1%) | 2 |
| Lip swelling | 0 | 0 | 0 | 0 | 1(2%) | 1 | 1(1%) | 1 |
| Mucous stools | 0 | 0 | 1(2%) | 1 | 0 | 0 | 1(1%) | 1 |
| Nausea | 0 | 0 | 1(2%) | 1 | 0 | 0 | 1(1%) | 1 |
| Toothache | 0 | 0 | 0 | 0 | 1(2%) | 1 | 1(1%) | 1 |
| Musculoskeletal and connective | 5(10%) | 5 | 4(8%) | 7 | 4(8%) | 4 | 13(8%) | 16 |
| tissue disorders | | | | | | | | |
| Arthralgia | 0 | 0 | 2(4%) | 3 | 1(2%) | 1 | 3(2%) | 4 |
| Back pain | 1(2%) | 1 | 0 | 0 | 1(2%) | 1 | 2(1%) | 2 |
| Joint swelling | 0 | 0 | 0 | 0 | 1(2%) | 1 | 1(1%) | 1 |
| Musculoskeletal pain | 1(2%) | 1 | 0 | 0 | 0 | 0 | 1(1%) | 1 |
| Myalgia | 0 | 0 | 0 | 0 | 1(2%) | 1 | 1(1%) | 1 |
| Myositis | 0 | 0 | 1(2%) | 1 | 0 | 0 | 1(1%) | 1 |
| Neck pain | 1(2%) | 1 | 0 | 0 | 0 | 0 | 1(1%) | 1 |
| Pain in extremity | 0 | 0 | 1(2%) | 1 | 0 | 0 | 1(1%) | 1 |
| Plantar fasciitis | 1(2%) | 1 | 1(2%) | 1 | 0 | 0 | 2(1%) | 2 |
| Synovial cyst | 1(2%) | 1 | 0 | 0 | 0 | 0 | 1(1%) | 1 |
| Tendonitis | 0 | 0 | 1(2%) | 1 | 0 | 0 | 1(1%) | 1 |
| Nervous system disorders | 5(10%) | 7 | 2(4%) | 2 | 5(10%) | 6 | 12(8%) | 15 |
| Amnesia | 0 | 0 | 0 | 0 | 1(2%) | 1 | 1(1%) | 1 |
| Dizziness | 1(2%) | 1 | 0 | 0 | 0 | 0 | 1(1%) | 1 |
| Headache | 4(8%) | 5 | 2(4%) | 2 | 3(6%) | 4 | 9(6%) | 11 |
| Paraesthesia | 1(2%) | 1 | 0 | 0 | 0 | 0 | 1(1%) | 1 |
| Sciatica | 0 | 0 | 0 | 0 | 1(2%) | 1 | 1(1%) | 1 |
| General disorders and administration | 2(4%) | 2 | 6(12%) | 6 | 1(2%) | 1 | 9(6%) | 9 |
| site conditions | | | | | | | | |
| Chest pain | 0 | 0 | 1(2%) | 1 | 0 | 0 | 1(1%) | 1 |
| Condition aggravated | 0 | 0 | 1(2%) | 1 | 0 | 0 | 1(1%) | 1 |
| Fatigue | 1(2%) | 1 | 3(6%) | 3 | 0 | 0 | 4(3%) | 4 |
| Inflammation | 1(2%) | 1 | 0 | 0 | 0 | 0 | 1(1%) | 1 |
| Pyrexia | 0 | 0 | 1(2%) | 1 | 0 | 0 | 1(1%) | 1 |
| Sensation of foreign body | 0 | 0 | 0 | 0 | 1(2%) | 1 | 1(1%) | 1 |
| Skin and subcutaneous tissue | 4(8%) | 4 | 2(4%) | 3 | 2(4%) | 2 | 8(5%) | 9 |
| disorders | | | | | | | | |
| Alopecia | 1(2%) | 1 | 0 | 0 | 0 | 0 | 1(1%) | 1 |
| Eczema | 1(2%) | 1 | 1(2%) | 1 | 0 | 0 | 2(1%) | 2 |
| Hyperhidrosis | 0 | 0 | 1(2%) | 1 | 0 | 0 | 1(1%) | 1 |
| Petechiae | 1(2%) | 1 | 0 | 0 | 0 | 0 | 1(1%) | 1 |
| Pruritus | 0 | 0 | 1(2%) | 1 | 1(2%) | 1 | 2(1%) | 2 |
| Rash | 0 | 0 | 0 | 0 | 1(2%) | 1 | 1(1%) | 1 |
| Urticaria | 1(2%) | 1 | 0 | 0 | 0 | 0 | 1(1%) | 1 |
| Metabolism and nutrition disorders | 3(6%) | 3 | 2(4%) | 2 | 1(2%) | 2 | 6(4%) | 7 |
| Diabetes mellitus | 0 | 0 | 1(2%) | 1 | 1(2%) | 1 | 2(1%) | 2 |
| Food craving | 1(2%) | 1 | 0 | 0 | 0 | 0 | 1(1%) | 1 |
| Gout | 1(2%) | 1 | 0 | 0 | 0 | 0 | 1(1%) | 1 |
| Hyperglycaemia | 0 | 0 | 0 | 0 | 1(2%) | 1 | 1(1%) | 1 |
| Increased appetite | 1(2%) | 1 | 1(2%) | 1 | 0 | 0 | 2(1%) | 2 |
| Respiratory, thoracic and mediastinal | 2(4%) | 2 | 3(6%) | 3 | 0 | 0 | 5(3%) | 5 |
| disorders | | | | | | | | |
| Cough | 1(2%) | 1 | 1(2%) | 1 | 0 | 0 | 2(1%) | 2 |
| Oropharyngeal pain | 1(2%) | 1 | 2(4%) | 2 | 0 | 0 | 3(2%) | 3 |
| Ear and labyrinth disorders | 1(2%) | 1 | 0 | 0 | 2(4%) | 2 | 3(2%) | 3 |
| Otolithiasis | 1(2%) | 1 | 0 | 0 | 0 | 0 | 1(1%) | 1 |
| Vertigo | 0 | 0 | 0 | 0 | 1(2%) | 1 | 1(1%) | 1 |
| Vertigo positional | 0 | 0 | 0 | 0 | 1(2%) | 1 | 1(1%) | 1 |
| Injury, poisoning and procedural | 1(2%) | 1 | 1(2%) | 1 | 1(2%) | 1 | 3(2%) | 3 |
| complications | | | | | | | | |
| Road traffic accident | 1(2%) | 1 | 0 | 0 | 1(2%) | 1 | 2(1%) | 2 |
| Thermal burn | 0 | 0 | 1(2%) | 1 | 0 | 0 | 1(1%) | 1 |
| Psychiatric disorders | 0 | 0 | 0 | 0 | 3(6%) | 4 | 3(2%) | 4 |
| Depression | 0 | 0 | 0 | 0 | 2(4%) | 2 | 2(1%) | 2 |
| Hallucination, auditory | 0 | 0 | 0 | 0 | 1(2%) | 1 | 1(1%) | 1 |
| Major depression | 0 | 0 | 0 | 0 | 1(2%) | 1 | 1(1%) | 1 |
| Reproductive system and breast | 0 | 0 | 1(2%) | 1 | 2(4%) | 2 | 3(2%) | 3 |
| disorders | | | | | | | | |
| Haematospermia | 0 | 0 | 0 | 0 | 1(2%) | 1 | 1(1%) | 1 |
| Menometrorragia | 0 | 0 | 1(2%) | 1 | 0 | 0 | 1(1%) | 1 |
| Ovarian cyst | 0 | 0 | 0 | 0 | 1(2%) | 1 | 1(1%) | 1 |
| Eye disorders | 1(2%) | 1 | 0 | 0 | 1(2%) | 1 | 2(1%) | 2 |
| Blepharitis | 0 | 0 | 0 | 0 | 1(2%) | 1 | 1(1%) | 1 |
| Retinal detachment | 1(2%) | 1 | 0 | 0 | 0 | 0 | 1(1%) | 1 |
| Investigations | 1(2%) | 3 | 1(2%) | 2 | 0 | 0 | 2(1%) | 5 |
| Alanine aminotransferase increased | 1(2%) | 1 | 1(2%) | 1 | 0 | 0 | 2(1%) | 2 |
| Aspartate aminotransferase | 1(2%) | 1 | 1(2%) | 1 | 0 | 0 | 2(1%) | 2 |
| increased | | | | | | | | |
| Gamma-glutamyltransferase | 1(2%) | 1 | 0 | 0 | 0 | 0 | 1(1%) | 1 |
| Surgical and medical procedures | 2(4%) | 3 | 0 | 0 | 0 | 0 | 2(1%) | 3 |
| Knee operation | 1(2%) | 1 | 0 | 0 | 0 | 0 | 1(1%) | 1 |
| Skin neoplasm excision | 1(2%) | 1 | 0 | 0 | 0 | 0 | 1(1%) | 1 |
| Toe operation | 1(2%) | 1 | 0 | 0 | 0 | 0 | 1(1%) | 1 |
| Cardiac disorders | 1(2%) | 1 | 0 | 0 | 0 | 0 | 1(1%) | 1 |
| Palpitations | 1(2%) | 1 | 0 | 0 | 0 | 0 | 1(1%) | 1 |
| Renal and urinary disorders | 0 | 0 | 0 | 0 | 1(2%) | 1 | 1(1%) | 1 |
| Hypertonic bladder | 0 | 0 | 0 | 0 | 1(2%) | 1 | 1(1%) | 1 |
| Vascular disorders | 0 | 0 | 1(2%) | 1 | 0 | 0 | 1(1%) | 1 |
| Hypertension | 0 | 0 | 1(2%) | 1 | 0 | 0 | 1(1%) | 1 |

The AE “back pain due to traffic accident” was coded with multiple MedDRA terms and is represented as separate AEs in the table n, number of subjects; m, number of events. Percentages are based on the number of subjects randomized. Baseline events (events that occurred prior to first dose) are omitted from summary.

TABLE A14
GI tolerability (GSRS) at baseline and absolute
changes after 2, 4, 7, 8, 14 and 26 weeks.

| | |
|---|---|
| P-value, | P-value, |
| EMP16- | EMP16- |

| | | | | |
|---|---|---|---|---|
| EMP16 | 120/40 vs | EMP16 | 150/50 vs | |
| 120 mg/40 mg | placebo | 150 mg/50 mg | placebo | PLACEBO |

Diarrhoea Syndrome

| | | | | | | |
|---|---|---|---|---|---|---|
| Baseline | 1.4 (0.7) | | 1.4 | (0.7) | | 1.2 (0.4) |
| End of week 2 | 1.3 (1.3) | 0.001 | 1.4 | (1.2) | <0.001 | 0.4 (0.8) |
| End of week 4 | 1.6 (1.4) | <0.001 | 1.6 | (1.1) | <0.001 | 0.5 (0.8) |
| Week 7 | 1.9 (1.3) | <0.001 | 2.1 | (1.4) | <0.001 | 0.5 (1.0) |
| Week 8 | 1.8 (1.3) | <0.001 | 2.2 | (1.5) | <0.001 | 0.6 (0.8) |
| Week 14 | 1.8 (1.6) | <0.001 | 2.2 | (1.4) | <0.001 | 0.3 (0.6) |
| Week 26 | 1.3 (1.5) | <0.001 | 1.8 | (1.4) | <0.001 | 0.3 (0.8) |

Indigestion syndrome

| | | | | | | |
|---|---|---|---|---|---|---|
| Baseline | 1.9 (0.8) | | 1.9 | (0.7) | | 1.8 (0.6) |
| End of week 2 | 1.0 (0.9) | <0.001 | 1.1 | (1.0) † | <0.001 | 0.3 (0.8) |
| End of week 4 | 0.9 (1.0) | <0.001 | 1.2 | (1.0) † | <0.001 | 0.2 (0.7) |
| Week 7 | 1.2 (1.0) | <0.001 | 1.3 | (1.1) † | <0.001 | 0.3 (0.7) |
| Week 8 | 1.0 (1.1) | 0.002 | 1.1 | (0.9) ‡ | <0.001 | 0.4 (0.8) |
| Week 14 | 0.9 (1.1) | 0.001 | 1.3 | (1.0) † | <0.001 | 0.3 (0.8) |
| Week 26 | 0.8 (1.0) | <0.001 | 1.0 | (1.1) † | <0.001 | 0.1 (0.7) |

Constipation Syndrome

| | | | | | | |
|---|---|---|---|---|---|---|
| Baseline | 1.5 (0.8) | | 1.3 | (0.5) | | 1.2 (0.4) |
| End of week 2 | 0.1 (0.8) | 0.005 | 0.4 | (0.9) | 0.366 | 0.6 (1.2) |
| End of week 4 | 0.3 (0.7) | 0.113 | 0.3 | (0.8) | 0.066 | 0.6 (0.9) |
| Week 7 | 0.5 (1.0) | 0.645 | 0.4 | (1.0) | 0.726 | 0.4 (0.6) |
| Week 8 | 0.2 (1.0) | 0.086 | 0.4 | (0.7) | 0.472 | 0.5 (0.9) |
| Week 14 | 0.3 (1.0) | 0.344 | 0.4 | (0.8) | 0.541 | 0.5 (0.8) |
| Week 26 | 0.2 (1.0) | 0.831 | 0.3 | (0.6) | 0.404 | 0.1 (0.5) |

Abdominal pain Syndrome

| | | | | | | |
|---|---|---|---|---|---|---|
| Baseline | 1.5 (0.6) | | 1.6 | (0.6) | | 1.5 (0.5) |
| End of week 2 | 0.3 (0.8) | 0.126 | 0.1 | (0.8) | 0.885 | 0.1 (0.6) |
| End of week 4 | 0.4 (0.8) | 0.039 | 0.2 | (0.7) | 0.754 | 0.1 (0.7) |
| Week 7 | 0.5 (0.8) | 0.016 | 0.2 | (0.9) | 0.513 | 0.1 (0.6) |
| Week 8 | 0.4 (0.9) | 0.161 | 0.1 | (1.0) | 0.710 | 0.2 (0.6) |
| Week 14 | 0.4 (0.9) | 0.156 | 0.5 | (1.0) | 0.041 | 0.1 (0.5) |
| Week 26 | 0.2 (0.7) | 0.079 | 0.1 | (0.8) | 0.284 | −0.1 (0.6) |

Reflux Syndrome

| | | | | | | |
|---|---|---|---|---|---|---|
| Baseline | 1.3 (0.7) | | 1.3 | (0.5) | | 1.2 (0.4) |
| End of week 2 | 0.1 (0.7) | 0.279 | 0.0 | (0.6) | 0.234 | 0.2 (0.7) |
| End of week 4 | 0.2 (0.9) | 0.954 | 0.0 | (0.7) | 0.127 | 0.2 (0.6) |
| Week 7 | 0.3 (0.7) | 0.860 | 0.0 | (0.6) | 0.039 | 0.3 (0.5) |
| Week 8 | 0.1 (1.0) | 0.705 | 0.1 | (0.6) | 0.633 | 0.2 (0.6) |
| Week 14 | 0.1 (0.9) | 0.402 | 0.1 | (0.7) | 0.345 | 0.2 (0.7) |
| Week 26 | 0.0 (0.9) | 0.1105 | −0.1 | (0.4) | 0.0666 | 0.2 (0.5) |

TABLE A15
Cumulative drop-out rate

| | | | | | |
|---|---|---|---|---|---|
| | EMP16 | Chi-Square | EMP16 | Chi-Square | |
| | 120 mg | p-value vs | 150 mg | p-value vs | |
| Visits | 40 mg | placebo | 50 mg | placebo | Placebo |

GI related withdrawal

| | | | | | |
|---|---|---|---|---|---|
| Visit 2 (week 1) | 0(0.0%)/52¹ | NE | 0(0.0%)/52 | NE | 0(0.0%)/52 |
| End of week 2 | 0(0.0%)/52 | NE | 1(1.9%)/52 | 0.315 | 0(0.0%)/52 |
| End of week 4 | 1(1.9%)/52 | 0.315 | 1(1.9%)/52 | 0.315 | 0(0.0%)/52 |
| Visit 3 (week 7) | 2(3.8%)/52 | 0.153 | 1(1.9%)/52 | 0.315 | 0(0.0%)/52 |
| End of week 8 | 3(5.8%)/52 | 0.079 | 2(3.8%)/52 | 0.153 | 0(0.0%)/52 |
| Visit 4 (week 14) | 4(7.7%)/52 | 0.041 | 4(7.7%)/52 | 0.041 | 0(0.0%)/52 |
| Visit 5 (week 26) | 4(7.7%)/52 | 0.041 | 5(9.6%)/52 | 0.022 | 0(0.0%)/52 |
| Visit 6 (week 28) | 4(7.7%)/52 | 0.041 | 5(9.6%)/52 | 0.022 | 0(0.0%)/52 |

Withdrawal overall

| | | | | | |
|---|---|---|---|---|---|
| Visit 2 (week 1) | 0(0.0%)/52 | NE | 0(0.0%)/52 | NE | 0(0.0%)/52 |
| End of week 2 | 0(0.0%)/52 | NE | 1(1.9%)/52 | 0.315 | 0(0.0%)/52 |
| End of week 4 | 1(1.9%)/52 | 0.315 | 1(1.9%)/52 | 0.315 | 0(0.0%)/52 |
| Visit 3 (week 7) | 3(5.8%)/52 | 0.308 | 1(1.9%)/52 | 1.000 | 1(1.9%)/52 |
| End of week 8 | 4(7.7%)/52 | 0.400 | 2(3.8%)/52 | 1.000 | 2(3.8%)/52 |
| Visit 4 (week 14) | 7(13.5%)/52 | 0.183 | 5(9.6%)/52 | 0.462 | 3(5.8%)/52 |
| Visit 5 (week 26) | 8(15.4%)/52 | 0.374 | 7(13.5%)/52 | 0.539 | 5(9.6%)/52 |
| Visit 6 (week 28) | 8(15.4%)/52 | 0.566 | 7(13.5%)/52 | 0.767 | 6(11.5%)/52 |

¹n(%)/N, NE = Not evaluable.
In cases of NE a warning occurred due to non-Chi² distribution.
Subjects who miss one or several visits and who later re-enter the study were not considered drop-outs until they missed all remaining visits.

TABLE A16
Compliance. Mean (SD)

| | EMP16 120mg/ | EMP16 150 mg/ | PLACEBO |
|---|---|---|---|
| | 40 mg (n = 45) | 50 mg (n = 46) | (n = 49) |
| Compliance (%) | 94.82 (10.17) | 95.71 (5.17) | 93.46 (9.92) |

No differences between the groups (data not shown).

### Discussion of Results

Of the 156 randomised participants, 149 constituted the modified ITT population and were assessed for the primary endpoint, and 135 completed the 28-week trial period. The PP population was comprised of 122 participants across treatment groups. In total, 111 women and 45 men were randomised in the trial. There were no important differences between the three groups at baseline (table 1).

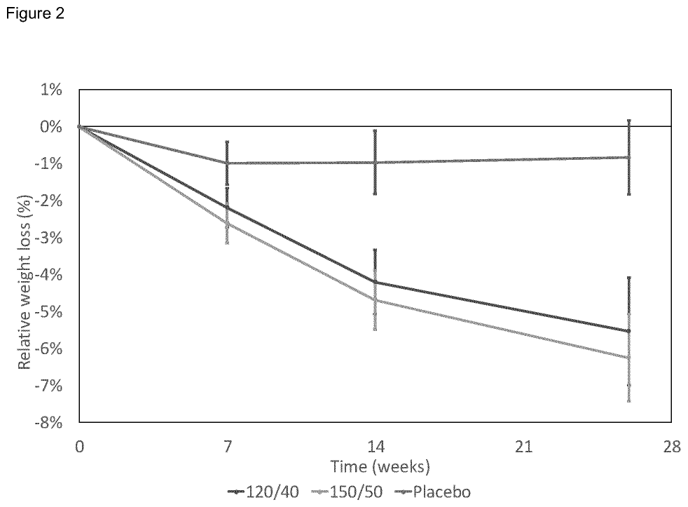

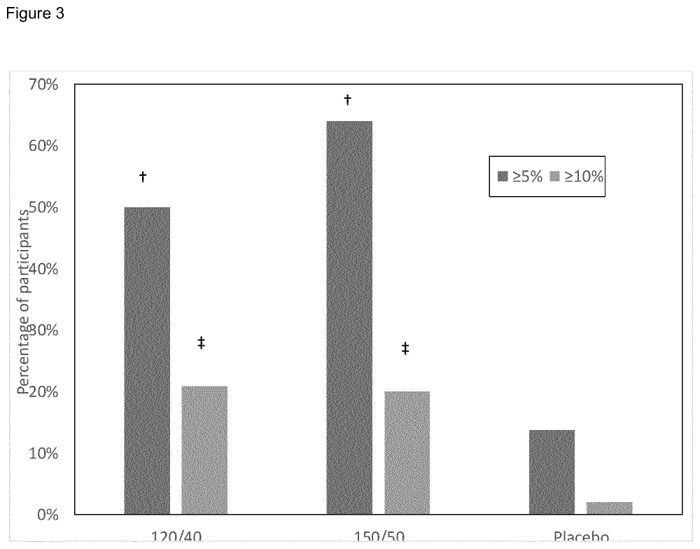

As illustrated in FIG. 2, participants treated with both doses of EMP16for 26 weeks lost more weight than those treated with placebo (p<0.0001). Mean relative weight loss in the ITT population was 5.53% with EMP16-120/40 as compared to 0.83% in the placebo group (estimated treatment difference −4.70 (95% confidence interval −6.16 to −3.24; table 2). More participants in the active treatment groups lost at least 5% and 10% of their baseline body weight at week 26 (FIG. 3). Similar weight loss was seen in the PP population or using the more conservative imputation method. Statistically significant absolute mean reductions in BMI and waist circumference were observed for participants treated with both EMP16 doses for 26 weeks as compared with placebo (table 3). The absolute mean sagittal diameter and body composition in terms of percentage body fat were significantly reduced in participants treated with EMP16-150/50 as compared with placebo, whereas the reductions in the EMP16-120/40 treatment group were not statistically significant.

There were no significant treatment differences in absolute mean changes from baseline in glucose metabolism markers (fasting glucose, insulin, HbA1c) or vital signs at week 26 (table 3). The lipid metabolism markers LDL and HDL cholesterol and total cholesterol, but not triglycerides, exhibited small but statistically significant reductions compared with the placebo group at week 26. There were no significant treatment differences in changes from baseline in T2DM or pre-diabetes status at week 26.

In general, there were no differences between the active treatment groups and the placebo group in the total scores for satiety and craving at week 26 (table 4).

Similarly, most participants appeared to follow the recommendations for healthy eating habits at baseline and no treatment differences in overall meal patterns were observed. Eating habits in relation to sweet food (cookies, chocolate, sweets, chips, soft drinks) and breakfast were improved in the EMP16-150/50 group, but not the EMP16-120/40 group, as compared to the placebo group at week 26.

Quality of life, based on the RAND-36 health survey, improved more in both active treatment groups compared to the placebo group between baseline and week 26 with respect to physical functioning, general health and the overall health transition score (table 4). In addition, participants in the EMP16-150/50 group improved more than those in the placebo group in terms of bodily pain, energy/fatigue and emotional well-being. There were no differences in activity and sleep habits between the study groups during the trial.

The mean scores in the GRSR diarrhoea and indigestion syndromes increased to a significantly greater extent in both active treatment groups compared to the placebo group at week 26 (table 4). Most participants rated their symptoms as mild or moderate. There were no treatment differences observed in the other parts of the GSRS.

A total of 191 AEs were reported by 101 (65%) of the 156 randomised participants with the three most common events being nasopharyngitis, diarrhoea and headache (table 5). Diarrhoea was reported only in the active treatment groups. Four of 52 participants (7.7%) in the EMP16-120/40 group and 5 of 52 participants (9.6%) in the EMP16-150/50 group withdrew early from the trial due to GI related AEs. In addition, 1 participant in the EMP-150/50 group withdrew consent to remain in the trial due to a COVID-19 infection. No participants in the placebo group withdrew due to an AE, whereas the overall withdrawal rate was comparable between the active treatment groups. Most AEs were mild or moderate in intensity. No deaths or serious AEs occurred during the trial. Compliance was high and no difference between the treatment groups was observed.

There were no clinically noteworthy changes in liver enzymes during the trial. A few individuals had a transient increase; however, these were not judged to be related to IMP according to the investigator, and the participants continued in the trial. There were no clinically relevant or statistically significant changes or differences in safety laboratory parameters or ECG during the trial (data not shown).

In this trial, treatment with EMP16 for 26 weeks led to a steady and clinically relevant weight loss. More than 50% of the participants in both active treatment groups lost at least 5% of their baseline weight, and more than 20% lost at least 10%, compared with 14% and 2% of participants, respectively, in the placebo group. Other anthropometric measurements such as BMI, waist circumference, sagittal abdominal diameter, and percentage of body fat showed similar treatment effects, albeit with larger effects for the higher EMP16-150/50 dose. Patient-reported quality of life showed improvements in both intervention groups, notably in physical functioning, general health, and the overall health transition score. Blood pressure, glucose metabolism markers and blood lipids were not notably affected, and no treatment differences were observed in the ratings of satiety and craving, although minor improvements in meal patterns in relation to sweet food and breakfast were seen in the EMP16-150/50 intervention group. EMP16 was generally well tolerated and no safety concerns were noted.

This trial corroborates the findings from the previous pilot trial with EMP16, which demonstrated that both the efficacy and tolerability of orlistat and acarbose were increased by employing a modified-release intervention. Orlistat and acarbose treatment in their conventional dosage forms typically provide an approximate relative weight loss of 2-3% and less than 0.5%, respectively, whereas the observed mean placebo-adjusted weight losses in the EMP16 arms were approximately 5%. The efficacy of EMP16 seems equivalent to most currently approved weight-loss drugs. Furthermore, a combination of orlistat and acarbose in their conventional dosage forms would likely cause tolerability problems and potentially augment their associated GI side effects such as flatulence with or without discharge. EMP16 was designed to encompass three contributing factors: (i) the mechanisms of action of orlistat and acarbose in their conventional dosage form on energy uptake; (ii) employment of a modified-release pattern to ensure that food-derived ligands are delivered to various appetite regulating checkpoints in the GI tract in an appropriate manner; and (iii) improved tolerability.

Despite the weight loss achieved with EMP16, there were limited effects on blood pressure, glucose metabolism and blood lipids. However, as evident from the baseline characteristics, this was a fairly healthy population of individuals with obesity. The prevalence of hypertension at baseline was approximately 30%, whereas a prevalence of about 60% is typically observed in patients with obesity. Furthermore, baseline blood lipid levels were generally low in the current study, with a small proportion of individuals (7%) on blood lipid medication, and most participants having a baseline HbA1c value below 40 mmol/mol. The observed lack of clinically relevant changes in metabolic risk markers is in line with similar weight loss studies in patients with obesity without diabetes using liraglutide or semaglutide.

Patient-reported quality of life was clearly improved in the intervention groups, especially in the EMP16 150/50 group, with distinct clinically relevant differences in several of the RAND-36 domains. The differences recorded were larger than seen in other weight loss studies using orlistat in its conventional dosage form, or liraglutide.

The larger increase in rated quality of life was somewhat unexpected, and in an exploratory post-hoc analyses a connection between weight loss and increased rated health can be seen. However, as the explained variance was quite low, additional factors apart from weight loss must have been involved in the increased quality of life ratings.

There were no significant treatment differences in the ratings of satiety and craving. In the pilot study, satiety was greater in the EMP16 groups compared to conventional orlistat. As stated earlier, orlistat administered in a conventional dosage form is associated with an increased appetite compared to placebo, partly by its effect on satiety sensing cells in the duodenum. With the MR preparation employed in EMP16, the orlistat-mediated effect on appetite seems to be reduced.

No large treatment differences were observed in reported meal patterns although participants in the EMP16-150/50 group reported increased breakfast intake and decreased intake of sweets and cakes. This could possibly be a “nudging” effect of EMP16, as side-effects are worsened if sweet, high-fat meal items are consumed, and the “cost” of not eating correctly appeared higher in the EMP16-150/50 group. Alternatively, EMP16 may have triggered an incretin effect, which affected the participants preference for sweets and cakes. We did not observe any effects on GLP-1 in the previous 14-day pilot trial. However, it possibly takes longer and higher doses to elicit an incretin response in participants with obesity.

Orlistat and acarbose in their conventional dosage forms are associated with frequent GI side effects, which limit their popularity. In the present trial, 15% of the participants receiving EMP16 reported diarrhoea whereas no participants in the placebo group did so, and more subjects (6-8%) in the EMP16 groups reported flatulence compared to the placebo group (2%). Since GI events were recorded as AEs in the trial only if they were judged by the investigator as being severe or leading to withdrawal, some cases of minor or moderate GI-events were not registered. The results from the GSRS corroborate that the frequency of diarrhoea with EMP16 was greater than that in the placebo group, but the diarrhoea syndrome scores in both intervention groups were around 3 (mild discomfort). The participants in the active groups also rated the GSRS indigestion syndrome as mild discomfort. Below 2 (minor discomfort) is usually indicative of normal gastric function.^{18,28} Faecal incontinence is perhaps the most problematic side effect associated with conventional orlistat but this seemed not to be an issue in the present trial with only one reported event, albeit of severe intensity, in the EMP16-150/50 group. In a similar 6-month trial, 5% of participants in the conventional orlistat arm reported faecal incontinence, whereas approximately 18% of participants reported such events in trials of longer duration. Lastly, only one participant reported nausea, and none reported vomiting, which is in contrast to studies using liraglutide.

This was a proof-of-concept trial with limited interaction between trial sites and participants; there were few visits with limited activities. Moreover, the participants did not receive any life-style instructions, only limited information regarding dietary choices. In contrast to many weight-loss studies, a run-in participant selection procedure before randomisation was not used. One reason for this “lean” design was to mimic a real-life situation and better understand the efficacy of EMP16 in a setting more closely resembling a clinical situation, somewhat analogous to a phase IV trial. The chosen design might explain the limited placebo effect, and ensured that the trial could be conducted during the COVID-19 pandemic without any major interruptions. Only a few participants had their last visit postponed for more than a week.

One limitation of the trial, not unique to EMP16, is the potential problem of maintaining masking, in particular since one of the known side effects of conventional orlistat is oily stools. Participants guessing that they had received placebo may have had a lower motivation to fulfil the additional lifestyle instructions, which could have led to an increased difference in outcomes between the active treatments and placebo. However, there were no differences in compliance between the treatment groups and the withdrawal rate for all groups was low (≤15%).

Another weakness of the trial was the chosen imputation method. The LOCF imputation method was prespecified in the protocol and has previously been recommended by regulatory authorities but is no longer regarded as optimal. A more conservative imputation method was added post-hoc, and comparable results were obtained.

Overall, this trial supports that orlistat and acarbose can be successfully combined as a promising potential candidate for improved weight management. The magnitude of the weight loss may have been less than that achieved with semaglutide. However, as stated in the Obesity Canada guidelines, “The individual response to obesity management pharmacotherapy is heterogeneous; the response to medications can differ from patient to patient”, thus more tools in the toolbox can only benefit the individual with obesity. Furthermore, obesity is a chronic disease and may require long-term treatment. Both orlistat and acarbose in their conventional dosage forms have already demonstrated long-term safety. No safety issues were observed in the present trial with EMP16, and in general the safety and tolerability of the modified release drug product appeared to be improved compared to the conventional products. The efficacy and safety of EMP16 remain to be evaluated in a study of longer duration in a more diverse population.

### Quality of Life

### RAND-36

The RAND-36 health questionnaire comprises 36 questions. The questionnaire taps eight health concepts: physical functioning, bodily pain, role limitations due to physical health problems, role limitations due to personal or emotional problems, emotional well-being, social functioning, energy/fatigue, and general health perception. It also includes a single item that provides an indication of perceived change in health. A low score indicates poor health-related quality of life while a high score indicates good health-related quality of life.

In the EMP-120/40 group, a mean absolute increase ≥3 points from baseline to week 26 was seen in 3 out of the 8 domains (physical functioning, role limitations due to physical health problems and general health). Similarly, the mean score increased by ≥10% from baseline in 4 out of the 8 domains (physical functioning, role limitations due to physical health problems, general health and energy/fatigue). The mean overall health transition score increased by 18.5 points, corresponding to a relative increase of 41.3%.

The differences between the EMP-120/40 group and the placebo group were statistically significant for the domains physical functioning (p=0.0076 [absolute change from baseline] and p=0.0131 [relative change from baseline]), general health (p=0.0171 [absolute change from baseline] and p=0.0180 [relative change from baseline]) and the mean overall health transition score (p=0.0058 [absolute change from baseline] and p=0.0138 [relative change from baseline]), see Table 14.3-35. In the EMP-150/50 group, a mean absolute increase by ≥3 points from baseline to week was seen in in 7 out of the 8 domains (physical functioning, role limitations due to physical health problems, bodily pain, general health, energy/fatigue, social functioning and role limitations due to emotional problems). Similarly, the mean score increased by ≥10% from baseline in all domains, except emotional well-being. The mean overall health transition score increased by 16.5 points, corresponding to a relative increase of 48.4%.

The differences between the EMP-150/50 group and the placebo group were statistically significant for physical functioning (p=0.0019 [absolute change from baseline] and p=0.0024 [relative change from baseline]), pain (p=0.0134 [absolute change from baseline] and p=0.0278 [relative change from baseline]), general health (p=0.0003 [absolute change from baseline] and p=0.0006 [relative change from baseline]), energy/fatigue (p=0.0224 [absolute change from baseline] and p=0.0287 [relative change from baseline]), emotional well-being (p=0.0363 [absolute change from baseline] and p=0.0390 [relative change from baseline]) and the mean overall health transition score (p=0.0111[absolute change from baseline] and p=0.0057 [relative change from baseline]).

In the placebo group, there was no increase in mean total score by_>3 points or by ≥10% from baseline in any domain. The largest positive change from baseline occurred in the domain physical functioning (mean absolute increase: 2.0 points, mean relative increase: 3.8%). The mean total score of the remaining domains were in the same range as at baseline, or lower, at week 26. Still, the mean overall health transition score increased by 5.9 points/13.0%.

### Conclusions

### Efficacy Conclusions

Twenty-six weeks of treatment with EMP16 in 2 doses (120 mg orlistat/40 mg acarbose or 150 mg orlistat/50 mg acarbose) had a significant and clinically relevant effect on body weight loss in obese patients. Patients treated with EMP16-120/40 or EMP16-150/50 lost 5.8%/-5.75 kg and 6.5%/-6.44 kg in body weight, respectively, while placebo patients lost 0.7%/-0.78 kg. Both doses of EMP16 gave rise to greater proportions of patients ≥5% and _≥10% weight loss and had a significant effect on reductions in BMI and waist circumference. The higher EMP16 dose also had significant effects on reductions in sagittal diameter and percentage body fat.
EMP16 did not induce any clinically relevant effects on fasting glucose metabolism markers, lipid metabolism markers or liver enzymes, had no apparent effect on the diabetic and prediabetic status of patients and no apparent effect on blood pressure compared to placebo, which is reasonably expected in a patient population with low prevalence of hypertension and with blood lipids and HbA1c within normal ranges as in the EP-002 study.
Both doses of EMP16 had significant positive effect on health-related quality of life. In general, there were no apparent differences between the treatment groups in terms of appetite and overall eating habits but EMP16-150/50 had a significant positive effect on the intake of sweet food and breakfast habits.

In this lean 6-month study, a clear synergistic effect was observed as the relative weight loss achieved with both doses of EMP16-02 were more than twice the expected weight loss; as estimated from of the numerical addition of the individual contribution of conventional orlistat and acarbose. Furthermore, the trend of the weight loss curve indicates that a nadir will not be reached until about 9-10 months. Modelling indicates that the EMP16-02 arms will possibly then be around 6.5-7.0% relative weight loss at 12 months, whereas the placebo group most probably will have returned to baseline. In addition, body composition showed a clear health benefit with decreased proportion of body fat, as well as decreased waist circumference and sagittal diameter. The latter two are indices of central adiposity, which is directly related to health outcomes. Moreover, the compliance was high, the number of gastrointestinal side-effects were low and there was a low level of dropouts. In this healthy obese population, there were little, which would be expected, changes in secondary outcomes from analyses of blood samples. The proven safety from all previous trials using orlistat and acarbose were confirmed with no SAE or AEs of concern. No untoward outcome in any safety variable was observed.

### SPECIFIC EMBODIMENTS

1. A composition of orlistat and acarbose for use in medicine, wherein the combination is administered orally to obtain a synergistic effect.
2. A composition for use according to item 1, wherein the synergistic effect is obtained after 13 weeks or more such a after 14 weeks or more, after 15 weeks or more, after 16 weeks or more, after 17 weeks or more, after 18 weeks or more, after 19 weeks or more, after 20 weeks or more, after 21 weeks or more, after 22 weeks or more, after 23 weeks or more, after 24 weeks or more, after 25 weeks or more of after 26 weeks or more of treatment.
3. A composition for use according to item 1 or 2, wherein the synergistic effect gives improved weight loss.
4. A composition for use according to item 3, wherein the improved weight loss is a weight loss that is at least 10% such as at least 20%, at least 30%, at least 40%, at least 50%, at least 60%, at least 70%, at least 80%, at least 90% or at least 100% more than a weight loss expected based on an additive effect of orlistat and acarbose.
5. A composition for use according to any one of the preceding items, wherein the synergistic effect results in a loss of body weight of a subject that is 3% or more such as about 4% or more, about 5% or more or about 6% based on the body weight of the subject before treatment.
6. A composition for use according to item 1, wherein the synergistic effect is obtained by administration of orlistat and acarbose in a weight ratio in a range of from 2:1 to 4:1 such as a weight ratio of 3:1.
7. A composition for use according to any one of the preceding items, wherein the combination is administered once daily, twice daily or three times daily.
8. A composition for use according to any one of the preceding items, wherein a daily dose of orlistat for an adult is 250 mg or more such as 270 mg or more 360 mg or more, 450 mg or more.
9. A composition for use according to any one of the preceding items, wherein a daily dose of acarbose for an adult is 75 mg or more such as 90 mg or more, 120 mg or more or 150 mg or more.
10. A composition for use according to any one of the preceding items, wherein a daily dose of orlistat for a child 5-10 years old weighing 40-60 kg is 120 mg, for a child 5-10 years old weighing 60-70 kg is 270 mg, and for a child older than 10 years old and/or weighing more than 70 kg is the same as for an adult.
11. A composition for use according to any one of the preceding items, wherein a daily dose of acarbose for a child 5-10 years old weighing 40-60 kg is 60 mg, for a child 5-10 years old weighing 60-70 kg is 90 mg, and for a child older than 10 years old and/or weighing more than 70 kg is the same as for an adult.
12. A composition for use according to any one of the preceding items comprising 90 mg orlistat/30 mg acarbose, 120 mg orlistat/40 mg orlistat or 150 mg orlistat/50 mg acarbose.
13. A composition for use according to any one of the preceding items, combination comprising granules, spheres or pellets.
14. A composition for use according to any one of the preceding items comprising enteric coated granules, spheres or pellets comprising orlistat.
15. A composition for use according to any one of the preceding items comprising enteric coated granules, spheres or pellets comprising acarbose.
16. A composition for use according to any one of the preceding items comprising modified release granules, sphere or pellets.
17. A composition for use according to any one of the preceding items comprising three or four different parts:
a) a first part, G1, comprising from about 45% w/w to about 65% w/w such as from about 50% w/w to about 65% w/w, from about 55% w/w to about 65% w/w or about 60% w/w of the total dose of acarbose,
b) a second part, G2A, comprising from about 35% w/w to about 55% w/w such as from about 35% w/w to about 50% w/w, from about 35% w/w to about 45% w/w or about 40% w/w of the total dose of acarbose,
c) a third part, G2B, comprising from about 50% w/w to about 85% w/w such as from about 55% w/w to about 80% w/w, from about 60% w/w to about 80% w/w, from about 65% w/w to about 75% w/w, from about 68% w/w to about 75% w/w, from about 72% w/w to about 73% w/w such as about 72.2% w/w of the total dose of orlistat, and
d) a fourth part, G3, comprising from about 15 to about 50% w/w such as from about 20% w/w to 40% w/w, from about 25% to about 35% w/w, from about 25% to about 32% w/w, from about 27% w/w to about 28% w/w or about 27.8% w/w of the total dose of orlistat, and the total concentration of acarbose and orlistat, respectively, is 100% w/w.
   18. A composition for use according to item 17 wherein part b) and c) are combined.
   19. A composition for use according to item 17 or 18, wherein the concentration of acarbose in the first part G1 is in a range of from 25% w/w to about 50% w/w such as from about 30% w/w to about 45% w/w or about 40% w/w based on the total weight of part G1.
   20. A composition for use according to any one of items 17-19, wherein the concentration of acarbose in the second part G2A or G2 is in a range of from about 0.5% w/w to about 4.5% w/w such as from about 1% w/w to about 4% w/w, from about 1.5% w/w to about 3.5% w/w, from about 2% w/w to about 3.5% w/w, from about 2.5% w/w to about 3.25% w/w or about 3% w/w based on the total weight of G2A or G2, whichever is relevant.
   21. A composition for use according to any one of items 17-20, wherein the concentration of orlistat in part G2B or G2 is in a range of from 5% w/w to about 30% w/w such as from about 10% w/w to about 25% w/w, from about 10% w/w to about 20% w/w, from about 12% w/w to about 20% w/w or about 15.5% w/w based on the total weight of G2B or G2, whichever is relevant.
   22. A composition for use according to any one of items 17-21, wherein the concentration of orlistat in part G3 is in a range of from 20% w/w to about 50% w/w such as from about 25% w/w to about 50% w/w, from about 30% w/w to about 45% w/w, from about 35% w/w to about 45% w/w or about 40% w/w based on the total weight of G3.
   23. A composition for use according to any one of items 17-22, wherein part G2 comprises a protective polymer in a concentration of at least 10% w/w such as in a range of from 10-20% w/w, from 12 to 20% w/w, from 13 to 20% w/w, from 13.5 to 20% w/w based on the weight of G2. G2A or G2B, whichever is relevant.
   24. A composition for use according to any one of the preceding items comprising modified release granules, spheres or pellets comprising orlistat, wherein the modified release granules, spheres or pellets contains from 30 to 50% w/w of orlistat.
   25. A composition for use according to any one of the preceding items comprising orlistat in micronized form, i.e., with an average particle size below 50 microns such as below 20 microns such as below 10 microns.
   26. A composition for use according to any one of the preceding items comprising modified release granules, spheres or pellets containing from 35 to 60% w/w of cellulose or a cellulose derivative such as microcrystalline cellulose.
   27. A composition for use according to any one of the preceding items comprising modified release granules, spheres or pellets comprising from 30 to 50% w/w of micronized orlistat, from 35 to 60% w/w of microcrystalline cellulose and from 10 to 18% w/w of polysorbate 80.
   28. A composition as defined in any one of items 17-27.
   29. A method for obtaining a synergistic effect of acarbose and orlistat, the method comprising administering to a subject a modified release composition comprising acarbose and orlistat to the subject.
   30. A method for obtaining improved body weight loss, the method comprising administering to a subject a modified release composition comprising acarbose and orlistat to the subject.

1. A composition comprising orlistat and acarbose for use in improving quality of life in obese or overweight subjects.

2. A composition comprising orlistat and acarbose for use in the treatment of obese or overweight subjects, which treatment leads to an improvement in quality of life.

3. A composition for use according to claim 1 or 2, wherein the obese or overweight subjects have a BMI of 27 kg/m² or more such as 29 kg/m² or more or 30 kg/m² or more.

4. A composition for use according to any one of the preceding claims comprising orlistat and acarbose in a weight ratio of from 2:1 to 4:1 such as 180/60 150/50 or 120/40 mg orlistat/mg acarbose.

5. A composition for use according to any one of the preceding claims, wherein improvement in quality of life is assessed by a questionnaire including questions to following domains: physical functioning, bodily pain, role limitations due to physical health problems, role limitations due to personal or emotional problems, emotional well-being, social functioning, energy/fatigue, and general health perception.

6. A composition for use according to any one of the preceding claims, wherein improvement in quality of life is assessed by RAND-36, SF-36, Impact of Weight on Quality of Life (IWQOL), Obesity and Weight Loss Quality of Life (OWLQOL) or similar health surveys.

7. A composition for use according to claim 5 or 6 measuring a mean overall health transition score.

8. A composition for use according to claim 7, wherein the improvement in mean overall health transition score is 10 points or more such as 12 points or more, 14 points or more, 15 points or more, 16 points or more compared with baseline.

9. A composition for use according to any one of claims 5-8, wherein the improvement is in at least 5 of the following domains: physical functioning, bodily pain, role limitations due to physical health problems, role limitations due to personal or emotional problems, emotional well-being, social functioning, energy/fatigue, and general health perception.

10. A composition for use according to any one of claims 5-9, wherein the improvement is measured as increased physical functioning and/or general health.

11. A composition for use according to claim 9 or 10, wherein the improvement in mean score is increased by 10 points or more compared to baseline.

12. A composition for use according to any one of claims 5-11, wherein the time period between answers is given to the questionnaire is at least 4 weeks such as at least 8 weeks.

13. A composition for use according to claim 12, wherein the time period is 26 weeks.

14. A composition for use according to any one of the preceding claims, wherein the improvement of quality of life is achieved regardless of achieved weight loss of said subject during treatment.

15. A composition for use according to any one of claims 1-13, wherein the improvement of quality of life is achieved in combination with an achieved weight loss of said subject during treatment of at least 5%.

16. A composition for use according to any one of the preceding claims, wherein the composition is administered one, two or three times daily.

17. A composition for use according to any one of the preceding claims, wherein a daily dose of orlistat in the treatment period is from 30 mg to 540 mg or more such as from 30 mg to 450 mg or more, from 60 mg to about 450 mg or more, from 90 mg to about 450 mg or more, from about 120 mg to 450 mg or more, from about 150 mg to about 450 mg or more, from 180 mg to 450 mg or more such as from 180 mg to 450 mg, from 270 mg to 450 mg, from 360 mg to 450 mg for an adult is 270 mg or more 360 mg or more or 450 mg or more.

18. A composition for use according to any one of the preceding claims, wherein a daily dose of acarbose in the treatment period is from 10 mg to about 180 mg such as from 10 mg to about 150 mg, from 20 mg to about 150 mg, from 30 mg to about 150 mg, from 40 mg to about 150 mg, from 50 mg to about 150 mg, from 60 mg to about 150 mg or more such as from 90 mg to 150 mg, 90 mg or more, 120 mg or more or 150 mg or more.

19. A composition for use according to any one the preceding claims, wherein said composition comprises 90 mg orlistat/30 mg acarbose, 120 mg orlistat/40 mg orlistat, 150 mg orlistat/50 mg acarbose or 180 mg orlistat/60 mg acarbose.

20. A composition for use according to any one of the preceding claims, wherein the treatment period is at least 2 weeks such as from about 2 weeks to about 1 year such as from about 2 weeks to about 9 months, from about 2 weeks to about 6 months, such as about 26 weeks, from about 2 weeks to about 5 months, from about 2 weeks to about 4 months, from about 2 weeks to about 3 months, from about 2 weeks to about 2 months.

21. A composition for use according to any one of the preceding claims, wherein the composition comprises granules, spheres or pellets.

22. A composition for use according to any one of the preceding claims, wherein the composition comprises orlistat in micronized form, i.e., with an average particle size below 50 microns such as below 20 microns such as below 10 microns.

23. A composition for use according to any one of the preceding claims, wherein the composition is an oral modified release composition comprises three or four different individual parts with different release pattern:
a) a first part, G1, comprising from about 45% w/w to about 65% w/w such as from about 50% w/w to about 65% w/w, from about 55% w/w to about 65% w/w or about 60% w/w of the total dose of acarbose,
b) a second part, G2A, comprising from about 35% w/w to about 55% w/w such as from about 35% w/w to about 50% w/w, from about 35% w/w to about 45% w/w or about 40% w/w of the total dose of acarbose,
c) a third part, G2B, comprising from about 50% w/w to about 85% w/w such as from about 55% w/w to about 80% w/w, from about 60% w/w to about 80% w/w, from about 65% w/w to about 75% w/w, from about 68% w/w to about 75% w/w, from about 72% w/w to about 73% w/w such as about 72.2% w/w of the total dose of orlistat, and
d) a fourth part, G3, comprising from about 15 to about 50% w/w such as from about 20% w/w to 40% w/w, from about 25% to about 35% w/w, from about 25% to about 32% w/w, from about 27% w/w to about 28% w/w or about 27.8% w/w of the total dose of orlistat,
and the total concentration of acarbose and orlistat, respectively, is 100% w/w,
wherein—when the composition contains three parts, the three parts are i) G1, ii) G2 wherein G2A and G2B are combined, and iii) G3.

24. A composition for use according to claim 23, wherein
i) part G1 is designed to release a part of the total dose of acarbose in the stomach,
ii) part G2A is designed to release a part of the total dose of acarbose in duodenum and jejunum; the release should be relatively fast, as acarbose should be available to exert their effect in duodenum and jejunum,
iii) part G2B is designed to release a part of the total dose of orlistat in duodenum and jejunum; the release should be relatively fast, as orlistat should be available to exert their effect in duodenum and jejunum, and
iv) part G3 is designed to release of a part of the total dose of orlistat in duodenum and jejunum.

25. A composition for use according to claim 23 or 24, wherein part b) and c) are combined.

26. A composition for use according to any one of claims 23-25, wherein G1 is in the form of inert cores coated with a composition comprising acarbose, G2A and G2B are combined to G2 and G2 is in the form of inert cores coated onto which acarbose and orlistat are applied and then provided with a coating with a protective polymer followed by coating with an enteric coating, and G3 is in the form of uncoated granules.

27. A composition for use according to claim 26, wherein the protective polymer is selected from cellulose, cellulose derivatives, and hydroxypropyl methylcellulose.

28. A composition for use according to any one of claims 23-27, wherein part G2A and G2B are combined to G2 and G2 comprises a protective polymer in a concentration of at least 10% w/w such as in a range of from 10-20% w/w, from 12 to 20% w/w, from 13 to 20% w/w, from 13.5 to 20% w/w based on the total weight of G2.

29. A composition for use according to any one of the preceding claims, wherein the composition comprises modified release granules, spheres or pellets comprising from 30 to 50% w/w of micronized orlistat, from 35 to 60% w/w of microcrystalline cellulose and from 10 to 18% w/w of polysorbate 80.

30. A composition for use according to any one of claims 23-28, wherein part G3 comprises modified release granules, spheres or pellets comprising from 30 to 50% w/w of micronized orlistat, from 35 to 60% w/w of microcrystalline cellulose and from 10 to 18% w/w of polysorbate 80.

31. A composition for use according to any one of claims 23-30, wherein the concentration of acarbose in the first part G1 is in a range of from 25% w/w to about 50% w/w such as from about 30% w/w to about 45% w/w or about 40% w/w based on the total weight of part G1.

32. A composition for use according to any one of claims 23-31, wherein the concentration of acarbose in the second part G2A or G2 is in a range of from about 0.5% w/w to about 4.5% w/w such as from about 1% w/w to about 4% w/w, from about 1.5% w/w to about 3.5% w/w, from about 2% w/w to about 3.5% w/w, from about 2.5% w/w to about 3.25% w/w or about 3% w/w based on the total weight of G2A or G2, whichever is relevant.

33. A composition for use according to any one of claims 23-32, wherein the concentration of orlistat in part G2B or G2 is in a range of from 5% w/w to about 30% w/w such as from about 10% w/w to about 25% w/w, from about 10% w/w to about 20% w/w, from about 12% w/w to about 20% w/w or about 15.5% w/w based on the total weight of G2B or G2, whichever is relevant.

34. A composition for use according to any one of claims 23-33, wherein the concentration of orlistat in part G3 is in a range of from 20% w/w to about 50% w/w such as from about 25% w/w to about 50% w/w, from about 30% w/w to about 45% w/w, from about 35% w/w to about 45% w/w or about 40% w/w based on the total weight of G3.

35. A composition according to any one of claims 23-34, wherein the composition comprises modified release granules, spheres or pellets containing from 35 to 60% w/w of cellulose or a cellulose derivative such as microcrystalline cellulose based on the total weight of the modified release granules, spheres or pellets.

36. Use of a composition comprising orlistat and acarbose to improve quality of life in obese or overweight subjects.

37. Use according to claim 30, wherein the composition is as defined in any one of claims 23-35.

38. Use according to claim 36 or 37, wherein the improvement of quality of life is as defined in any one of claims 1-15.
